(19) United States
(12) Patent Application Publication
(10) Pub. No.: US 2026/0265798 A1
(43) Pub. Date: Sep. 10, 2026

(54) Enzymes and Uses in Biocatalytic Halogenation of N-Heteroaryls Thereof

(71) Applicant: Agency for Science, Technology and Research, Singapore (SG)

(72) Inventors: Yee Hwee LIM, Singapore (SG); Ee Lui ANG, Singapore (SG); Fong Tian WONG, Singapore (SG); Guangrong PEH, Singapore (SG); Song Buck TAY, Singapore (SG)

(21) Appl. No.: 18/867,802

(22) PCT Filed: May 19, 2023
(86) PCT No.: PCT/SG2023/050347

§ 371 Date: Nov. 20, 2024

(30) Foreign Application Priority Data
May 20, 2022 (SG) 10202205361W

(51) Int. Cl. C12P 17/10; C07D 405/04; C12N 9/02; C12P 17/16

(52) U.S. Cl. CPC C12P 17/10; C07D 405/04; C12N 9/0071; C12P 17/165

(57) ABSTRACT

The present disclosure concerns enzymes and their uses in biocatalytic halogenation of pyrrolic heterocycles thereof. The method of halogenating a 2′, 3′ and/or 5′ substituted N-heteroaryl or a derivative thereof comprises contacting the N-heteroaryl or derivative thereof with a halogen and a flavin-dependent halogenase under co-enzyme regeneration conditions in order for the halogen to be substituted at a 4′ position on the N-heteroaryl or derivative thereof.

### TECHNICAL FIELD

The present invention relates, in general terms, to enzymes and their uses in biocatalytic halogenation of pyrrolic heterocycles thereof.

### BACKGROUND

Pyrroles are one of the privileged biological motif present in numerous natural products. Pyrrolic compounds have diverse uses in medicine and agrochemistry (antifungal, antibacterial, and anti-cancer) (FIG. 1), advanced materials (organic semiconductors, solar cells), and organic catalysts. The pyrrole ring is electron-rich and, when not part of a larger conjugated system, is susceptible to air oxidation. Many pyrrole natural products bear electron-withdrawing substituents, such as carbonyl, nitro and halides. Chemical halogenation of pyrroles is challenging due to their tendency to undergo uncontrolled poly-halogenation. Furthermore, due to the differing stability of the intermediates, pyrroles preferentially undergo C2 halogenation, making C3-selective halogenation difficult. (FIG. 2A).

It would be desirable to overcome or ameliorate at least one of the above-described problems.

### SUMMARY

The present invention provides a method of halogenating a 2′, 3′ and/or 5′ substituted N-heteroaryl or a derivative thereof, comprising:
a) contacting the N-heteroaryl or derivative thereof with a halogen and a flavin-dependent halogenase under co-enzyme regeneration conditions in order for the halogen to be substituted at a 4′ position on the N-heteroaryl or derivative thereof; wherein the co-enzyme regeneration conditions comprises flavin adenine dinucleotide (FAD) at about 0.01 equivalents to about 0.2 equivalents relative to a flavin-dependent halogenase concentration;

E. coli flavin reductase (Fre) at about 0.1 equivalents to about 0.5 equivalents relative to a flavin-dependent halogenase concentration;
nicotinamide adenine dinucleotide hydrogen (NADH) at about 2 equivalents to about 250 equivalents relative to a flavin-dependent halogenase concentration;
glucose 1-dehydrogenase (GdHi) at about 0.1 equivalents to about 0.5 equivalents relative to a flavin-dependent halogenase concentration;
monosaccharide at about 5 equivalents to about 500 equivalents relative to a flavin-dependent halogenase concentration.

In some embodiments, the co-enzyme regeneration conditions comprises NADH at about 0.05 equivalents to about 5 equivalents relative to a 2′, 3′ and/or 5′ substituted N-heteroaryl or a derivative thereof concentration.

In some embodiments, the co-enzyme regeneration conditions comprises monosaccharide at about 5 equivalents to about 20 equivalents relative to a 2′, 3′ and/or 5′ substituted N-heteroaryl or a derivative thereof concentration.

In some embodiments, a ratio of FAD:Fre:GDH is about 1:2.5:2.5.

In some embodiments, a ratio of FAD:Fre:NADH:GDH is about 1:2.5:2500:2.5.

In some embodiments, the co-enzyme regeneration conditions further comprises a halogen at about 10 equivalents to about 30 equivalents relative to a 2′, 3′ and/or 5′ substituted N-heteroaryl or a derivative thereof concentration.

In some embodiments, the halogen is derived from a halide, the halide selected from Cl⁻ or Br⁻.

In some embodiments, the co-enzyme regeneration conditions further comprises a phosphate buffer or a Tris HCl buffer.

In some embodiments, the flavin-dependent halogenases is monodechloroaminopyrrolnitrin halogenase (PrnC).

In some embodiments, the flavin-dependent halogenases comprises a N-terminal 11 amino acid solubility tag.

In some embodiments, the N-terminal 11 amino acid solubility tag is derived from a first 11 amino acid residues within a N-terminal N-half domain of a duplicated carbonic anhydrase (dCA) from Dunaliella species.

In some embodiments, the 11 amino acid solubility tag has an amino acid sequence of VSEPHDYNYEK.

In some embodiments, the N-heteroaryl or derivative thereof is a 5 membered N-heteroaryl or derivative thereof.

In some embodiments, the substituent on the 2′, 3′ and/or 5′ substituted N-heteroaryl or a derivative thereof is each selected from optionally substituted aryl or optionally substituted heteroaryl.

In some embodiments, the N-heteroaryl or derivative thereof is a compound of formula (I)

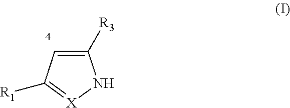

wherein X is selected from CR₂ or N;
R₁ is H, optionally substituted aryl, or optionally substituted heteroaryl;
R₂ is H, optionally substituted aryl, or optionally substituted heteroaryl;
R₃ is H, optionally substituted aryl, or optionally substituted heteroaryl;
wherein at least one of R₁, R₂ and R₃ is optionally substituted aryl or optionally substituted heteroaryl; or
R₁ and R₂ are linked to form optionally substituted aryl, or optionally substituted heteroaryl.

In some embodiments, when X is CR₂, R₃ is H.

In some embodiments, when X is CR₂, R₂ is H and R₃ is H.

In some embodiments, when X is N, R₁ is H.

In some embodiments, the N-heteroaryl or derivative thereof is selected from:

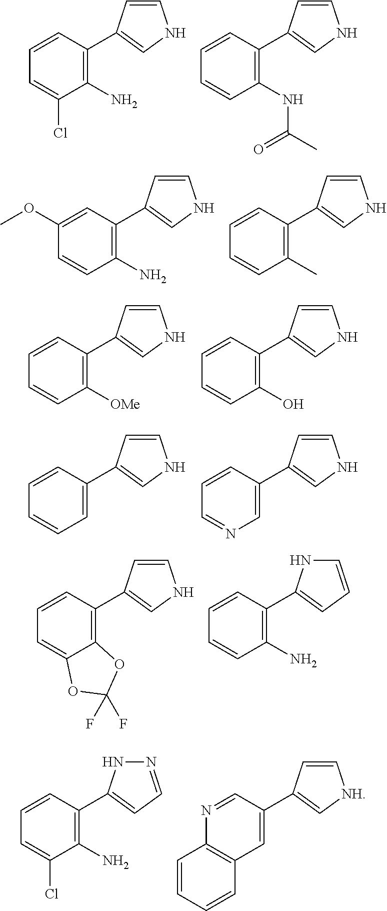

In some embodiments, the halogenated N-heteroaryl or derivative thereof is (where X₁ represents halo):

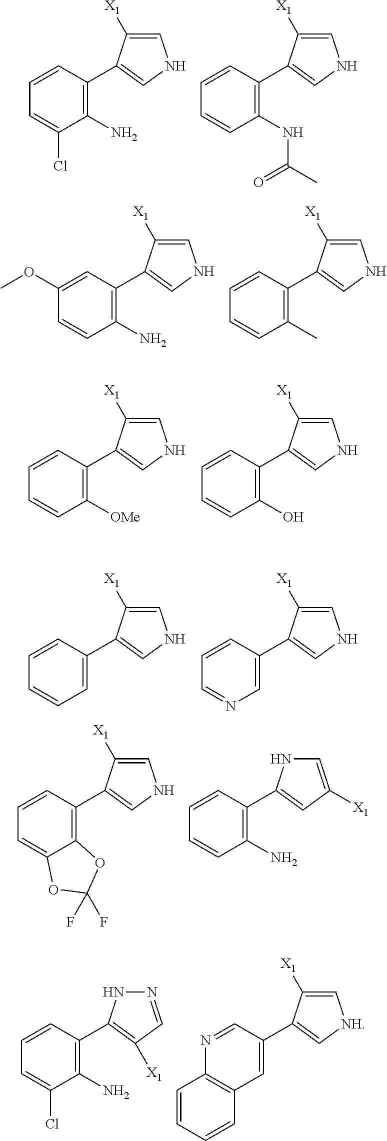

In some embodiments, the method is characterised by a regioisomeric ratio of 4′ substitution to 2′, 3′ or 5′ substitution is about 3:1 to about 1.1:1.

In some embodiments, the method is characterised by a ratio of 4′ monohalogenation to 2′, 4′ dihalogenation, 3′, 4′ dihalogenation, and/or 4′, 5′ dihalogenation of about 20:1 to about 4:1.

In some embodiments, the method is characterised by a K_{cat} of about 7×10⁻³ s⁻¹ to about 8×10⁻³ s⁻¹.

In some embodiments, the method is characterised by a Kₘ of about 1×10⁻⁵ M to about 2×10⁻⁵ M.

In some embodiments, the method is characterised by a K_{cat}/Kₘ of about 4×10² s⁻¹ M⁻¹ to about 5×10² s⁻¹ M⁻¹.

The present invention also provides a method of producing a flavin-dependent halogenase, comprising:
a) expressing the flavin-dependent halogenase in a cell;
wherein the flavin-dependent halogenase comprises an 11 amino acid solubility tag at a N-terminus thereof and a His6 tag at a C-terminus thereof.

In some embodiments, the method further comprises a step of purifying the flavin-dependent halogenase by metal affinity chromatography.

The present invention also provides a method of synthesising Fludioxonil, comprising:
a) contacting a compound of formula (II) or derivative thereof with a halogen and a flavin-dependent halogenase under co-enzyme regeneration conditions in order for the halogen to be substituted at a 4′ position on the compound or derivative thereof;

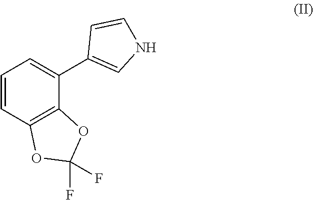 [SMILES: FC1(F)Oc2cccc(-c3cc[nH]c3)c2O1]

wherein the co-enzyme regeneration conditions comprises flavin adenine dinucleotide (FAD) at about 0.01 equivalents to about 0.2 equivalents relative to a flavin-dependent halogenase concentration;

E. coli flavin reductase (Fre) at about 0.1 equivalents to about 0.5 equivalents relative to a flavin-dependent halogenase concentration;
nicotinamide adenine dinucleotide hydrogen (NADH) at about 2 equivalents to about 250 equivalents relative to a flavin-dependent halogenase concentration;
glucose 1-dehydrogenase (GdHi) at about 0.1 equivalents to about 0.5 equivalents relative to a flavin-dependent halogenase concentration;
monosaccharide at about 5 equivalents to about 500 equivalents relative to a flavin-dependent halogenase concentration;
b) contacting the 4′ halogenated compound of step a) with a palladium precatalyst and a cyanide precursor.

### BRIEF DESCRIPTION OF THE DRAWINGS

Embodiments of the present invention will now be described, by way of non-limiting example, with reference to the drawings in which:

FIG. 1 shows examples of pyrrolic motifs in agrochemicals and natural products.

FIG. 2A-2C shows application of PrnC biocatalyst for the regioselective halogenation on the pyrrolic backbone.

FIGS. 3A and 3B shows preliminary optimisation results.

FIG. 4 shows conversion yields of in-vitro regioselective chlorination.

FIG. 5 shows a PrnC/MDA (1) complex model from the molecular modelling and molecular docking. K97, E129, and E60 are the proposed key residues.

FIG. 6 shows an application of the biocatalyst in the synthesis of agricultural and pharmaceutical drug molecules.

FIG. 7 shows an application of the biocatalyst in the synthesis of agricultural and pharmaceutical drug molecules. Fludioxonil is shown as an example. Without PrnC, no bromination occurred.

FIG. 8 shows Michaelis-Menten plot and table of kinetic parameters for PrnC chlorination of monodechloroaminopyrrolnitrin (1).

FIG. 9 shows a chart and table of MDA-Cl (2) product formation by PrnC enzyme variants; N.D.: not detectable.

FIG. 10 shows the sequence alignment and the template crystal structure of halogenase PItM (PDBCODE: 6BZA) were provided by BLASTP on the NCBI server.

FIG. 11 shows the native substrate MDA (1) in the binding pocket. Brown color refers to the hydrophobic residues; green color standing for the polar residues.

FIG. 12 shows calibration curves of MDA (1) and MDACI (2) for analytical HPLC.

FIG. 13 shows additional co-factors optimization parameters on 1 using NT11-PrnC.

FIG. 14 shows determination of optimum lysate amount for PrnC lysate runs.

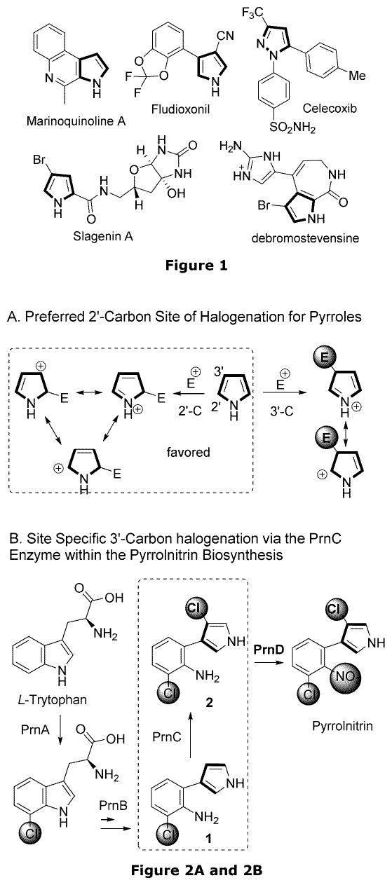

### DETAILED DESCRIPTION

The present disclosure is predicated on the understanding that in nature, aryl halides are usually installed by flavin-dependent halogenases. Many phenol and indole halogenases are known, and several have been applied in organic synthesis. Conversely, much fewer pyrrole halogenases are known, and their synthetic applications have not been investigated to date. Enzymatic pyrrole halogenation may offer mild reaction conditions, site-selective mono-halogenation without the need for any protective and/or directing groups, and avoids toxic halogenating reagents such as N-bromosuccinimide (NBS), iodine, and mercuric salts. In addition, the mono-halogenated site may provide a useful handle for late-stage functionalization through a plethora of metal-catalyzed coupling reactions.

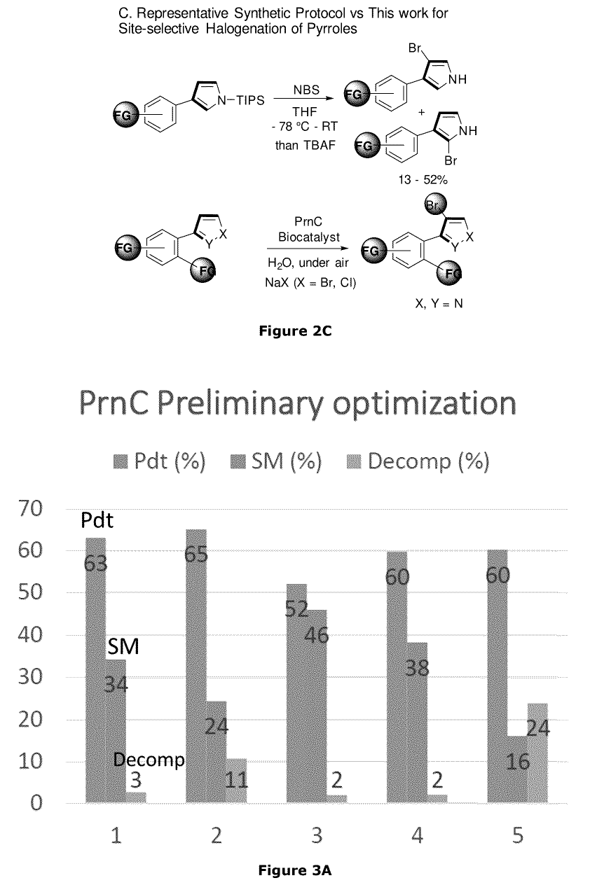

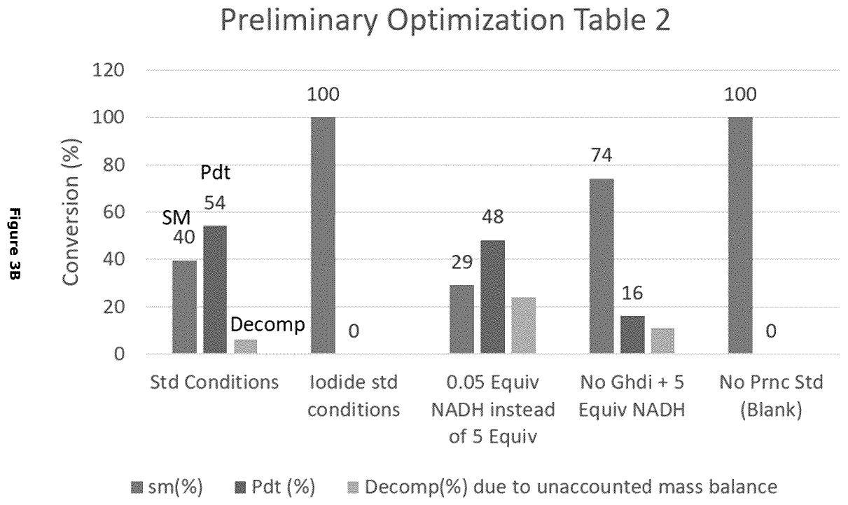

Of the known pyrrole halogenases, nearly all halogenate pyrrole forms a part of a biosynthetic intermediate that is tethered to a carrier protein. Monodechloroaminopyrrolnitrin 3-halogenase (PrnC) is the only flavin-dependent halogenase reported to act on a free-standing pyrrole substrate, making it a prime candidate for further investigations for synthetic applications. PrnC is part of the prnABCD cluster responsible for the biosynthesis of pyrrolnitrin (FIG. 2B). In the mechanism, the two chlorine atoms in pyrrolnitrin are introduced sequentially by the tryptophan halogenase PrnA, and the pyrrole halogenase PrnC, which is hypothesized to regioselectively chlorinate the C3 position of the pyrrolic backbone. While PrnA is well studied, there are no reports of heterologous expression of PrnC for biochemical study or in-vitro screening.

Without wanting to be bound by theory, the inventors believed that PrnC may be used to halogenate structurally diverse aryl or biaryl pyrroles. Towards this end, PrnC was characterized and a library of biaryl pyrroles were synthesized. The utility of the biocatalyst was also demonstrated in the synthesis of an agrochemical compound.

The present disclosure concerns a method of halogenating a 2′, 3′ and/or 5′ substituted N-heteroaryl or a derivative thereof, comprising:
a) contacting the N-heteroaryl or derivative thereof with a halogen and a flavin-dependent halogenase under co-enzyme regeneration conditions in order for the halogen to be substituted at a 4′ position on the N-heteroaryl or derivative thereof; wherein the co-enzyme regeneration conditions comprises:
i) flavin adenine dinucleotide (FAD) at about 0.01 equivalents to about 0.2 equivalents relative to a flavin-dependent halogenase concentration;
ii) E. coli flavin reductase (Fre) at about 0.1 equivalents to about 0.5 equivalents relative to a flavin-dependent halogenase concentration;
iii) nicotinamide adenine dinucleotide hydrogen (NADH) at about 2 equivalents to about 250 equivalents relative to a flavin-dependent halogenase concentration;
iv) glucose 1-dehydrogenase (GdHi) at about 0.1 equivalents to about 0.5 equivalents relative to a flavin-dependent halogenase concentration; and
v) monosaccharide at about 5 equivalents to about 500 equivalents relative to a flavin-dependent halogenase concentration.

In some embodiments, the N-heteroaryl or derivative thereof is a 5 membered N-heteroaryl or derivative thereof. Accordingly, the method concerns halogenation of a 2′, 3′ and/or 5′ substituted 5 membered N-heteroaryl or a derivative thereof.

In some embodiments, the substituent on the 2′, 3′ and/or 5′ substituted N-heteroaryl or a derivative thereof is each selected from optionally substituted aryl or optionally substituted heteroaryl.

In some embodiments, the N-heteroaryl or derivative thereof is a compound of formula (I)

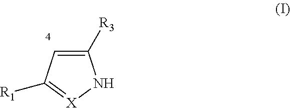

wherein X is selected from CR₂ or N;
R₁ is H, optionally substituted aryl, or optionally substituted heteroaryl;
R₂ is H, optionally substituted aryl, or optionally substituted heteroaryl;
R₃ is H, optionally substituted aryl, or optionally substituted heteroaryl;
wherein at least one of R₁, R₂ and R₃ is optionally substituted aryl or optionally substituted heteroaryl; or
R₁ and R₂ are linked to form optionally substituted aryl, or optionally substituted heteroaryl.

In some embodiments, X is CR₂. Accordingly, the compound of formula (I) may be

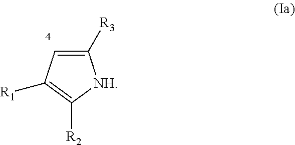

In some embodiments, X is N. Accordingly, the compound of formula (I) may be

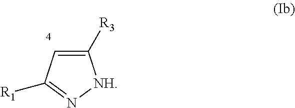

In some embodiments, at least one of R₁, R₂ and R₃ is optionally substituted aryl or optionally substituted heteroaryl. In some embodiments, the aryl or heteroaryl is independently 5 membered or 6 membered. In some embodiments, the aryl is phenyl. In some embodiments, the heteroaryl is selected from pyridinyl, benzodioxolyl or quinolinyl. The optional substituent may be an electron withdrawing group. The optional substituent may be substituted one or two times on the aryl or the heteroaryl. The optional substituent may be selected from halo, amino, alkyl, alkoxy, oxo, alkylacylamino, acyloxy, cycloalkyl, cycloalkenyl, or heterocyclyl.

In some embodiments, R₁ is optionally substituted aryl, or optionally substituted heteroaryl, and R₂ is H, optionally substituted aryl, or optionally substituted heteroaryl. In some embodiments, R₁ is optionally substituted aryl, or optionally substituted heteroaryl, and R₂ is H.

In some embodiments, R₂ is optionally substituted aryl, or optionally substituted heteroaryl, and R₁ is H, optionally substituted aryl, or optionally substituted heteroaryl. In some embodiments, R₂ is optionally substituted aryl, or optionally substituted heteroaryl, and R₁ is H.

In some embodiments, when X is CR₂, R₃ is H. In some embodiments, when X is CR₂, R₂ is H and R₃ is H.

In some embodiments, R₁ and R₂ are linked to form optionally substituted aryl, optionally substituted heteroaryl, optionally substituted cycloalkyl, or optionally substituted heterocyclyl. For example, the aryl, heteroaryl, cycloalkyl or heterocyclyl may be quinolinyl, aza-2-cycloheptanonyl, or aza-2-cycloheptanone-dienyl.

In some embodiments, when X is N, R₃ is optionally substituted aryl, or optionally substituted heteroaryl, R₁ is H, optionally substituted aryl, or optionally substituted heteroaryls. In some embodiments, when X is N, R₃ is optionally substituted aryl, or optionally substituted heteroaryl, R₁ is H. In some embodiments, when X is N, R₁ is H.

In some embodiments, the N-heteroaryl or derivative thereof is a compound of formula (Ia)

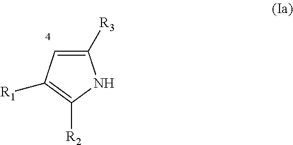

wherein R₁ is H, optionally substituted aryl, or optionally substituted heteroaryl;
R₂ is H, optionally substituted aryl, or optionally substituted heteroaryl; R₃ is H;
wherein at least one of R₁, and R₂ is optionally substituted aryl or optionally substituted heteroaryl; or
R₁ and R₂ are linked to form optionally substituted aryl, or optionally substituted heteroaryl.

In some embodiments, the N-heteroaryl or derivative thereof is a compound of formula (Ib)

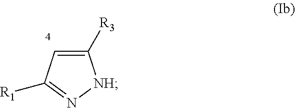

wherein R₁ is H; and
R₃ is optionally substituted aryl, or optionally substituted heteroaryl.

In some embodiments, when R₁, R₂ and R₃ are independently N-heteroaryl, the N-heteroaryl is substituted at its 4′ position. In some embodiments, R₁, R₂ and R₃ is not N-heteroaryl. In some embodiments, R₁, R₂ and R₃ is not 5 membered N-heteroaryl. In some embodiments, R₁, R₂ and R₃ is not pyrrolyl or pyrazolyl.

“Aryl” refers to an unsaturated aromatic carbocyclic group having a single ring (eg. phenyl) or multiple condensed rings (eg. naphthyl or anthryl), preferably having from 6 to 14 carbon atoms. Examples of aryl groups include phenyl, naphthyl and the like.

“Heteroaryl” refers to a monovalent aromatic heterocyclic group which fulfils the Hückel criteria for aromaticity (ie. contains 4n+2 n electrons) and preferably has from 2 to 10 carbon atoms and 1 to 4 heteroatoms selected from oxygen, nitrogen, selenium, and sulfur within the ring (and includes oxides of sulfur, selenium and nitrogen). Such heteroaryl groups can have a single ring (eg. pyridyl, pyrrolyl or N-oxides thereof or furyl) or multiple condensed rings (eg. indolizinyl, benzoimidazolyl, coumarinyl, quinolinyl, isoquinolinyl or benzothienyl).

Examples of heteroaryl groups include, but are not limited to, oxazole, pyrrole, imidazole, pyrazole, pyridine, pyrazine, pyrimidine, pyridazine, indolizine, isoindole, indole, indazole, purine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, thiazole, thiadiazoles, oxadiazole, oxatriazole, tetrazole, thiophene, benzo[b]thiophene, triazole, imidazopyridine and the like.

“Halo” or “halogen” refers to fluoro, chloro, bromo and iodo.

“Oxo/hydroxy” refers to groups ═O, HO—.

“Alkyl” refers to monovalent alkyl groups which may be straight chained or branched and preferably have from 1 to 10 carbon atoms or more preferably 1 to 6 carbon atoms. Examples of such alkyl groups include methyl, ethyl, n-propyl, iso-propyl, n-butyl, iso-butyl, n-hexyl, and the like.

“Alkoxy” refers to the group alkyl-O— where the alkyl group is as described above. Examples include, methoxy, ethoxy, n-propoxy, iso-propoxy, n-butoxy, tert-butoxy, sec-butoxy, n-pentoxy, n-hexoxy, 1,2-dimethylbutoxy, and the like.

“Amino” refers to the group —NR″R″ where each R″ is independently hydrogen, alkyl, cycloalkyl, aryl, heteroaryl, and heterocyclyl and where each of alkyl, cycloalkyl, aryl, heteroaryl and heterocyclyl is as described herein.

“Acylamino” refers to the group —NR″C(O) R″ where each R″ is independently hydrogen, alkyl, cycloalkyl, aryl, heteroaryl and heterocyclyl and where each of alkyl, cycloalkyl, aryl, heteroaryl, and heterocyclyl are as described herein.

“Acyloxy” refers to the groups-OC(O)-alkyl, —OC(O)-aryl, —C(O)O-heteroaryl, and —C(O)O-heterocyclyl where alkyl, aryl, heteroaryl and heterocyclyl are as described herein.

“Cycloalkyl” refers to cyclic alkyl groups having a single cyclic ring or multiple condensed rings, preferably incorporating 3 to 11 carbon atoms. Such cycloalkyl groups include, by way of example, single ring structures such as cyclopropyl, cyclobutyl, cyclopentyl, cyclohexyl, cyclooctyl, and the like, or multiple ring structures such as adamantanyl, indanyl, 1,2,3,4-tetrahydronaphthalenyl and the like.

“Cycloalkenyl” refers to cyclic alkenyl groups having a single cyclic ring or multiple condensed rings, and at least one point of internal unsaturation, preferably incorporating 4 to 11 carbon atoms. Examples of suitable cycloalkenyl groups include, for instance, cyclobut-2-enyl, cyclopent-3-enyl, cyclohex-4-enyl, cyclooct-3-enyl, indenyl and the like.

“Heterocyclyl” refers to a monovalent saturated or unsaturated group having a single ring or multiple condensed rings, preferably from 1 to 8 carbon atoms and from 1 to 4 hetero atoms selected from nitrogen, sulfur, oxygen, selenium or phosphorous within the ring. The most preferred heteroatom is nitrogen. It will be understood that where, for instance, R₂ or R′ is an optionally substituted heterocyclyl which has one or more ring heteroatoms, the heterocyclyl group can be connected to the core molecule of the compounds of the present invention, through a C—C or C-heteroatom bond, in particular a C—N bond.

Examples of heterocyclyl and heteroaryl groups include, but are not limited to, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, isothiazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiadiazoles, oxadiazole, oxatriazole, tetrazole, thiazolidine, thiophene, benzo[b]thiophene, morpholino, piperidinyl, pyrrolidine, tetrahydrofuranyl, triazole, and the like.

The halogenation occurs primarily at a 4′ position of the N-heteroaryl or a derivative thereof. For clarity, the 4′ position of a pyrrole and pyrazole is

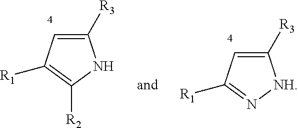

It was found that other isomers may form, but in lower quantities. In some embodiments, a regioisomeric ratio of 4′ substitution to 2′, 3′ or 5′ substitution is about 3:1 to about 1.1:1. In other embodiments, the ratio is about 3:1 to about 1.2:1, about 3:1 to about 1.3:1, about 3:1 to about 1.4:1, about 3:1 to about 1.5:1, about 2.5:1 to about 1.5:1, or about 2:1 to about 1.5:1.

In some embodiments, the method is characterised by a ratio of 4′ monohalogenation to dihalogenation of about 20:1 to about 4:1. The dehalogenation may occur at 2′ and 4′ position, 3′ and 4′ position, or 4′ and 5′ position. In other embodiments, the ratio is about 20:1 to about 5:1, about 20:1 to about 6:1, about 20:1 to about 7:1, or about 20:1 to about 10:1.

In some embodiments, the co-enzyme regeneration conditions comprises nicotinamide adenine dinucleotide hydrogen (NADH) at about 2 equivalents to about 250 equivalents relative to a flavin-dependent halogenase concentration. In other embodiments, the concentration is about 2 equivalents to about 200 equivalents, about 2 equivalents to about 150 equivalents, about 2 equivalents to about 100 equivalents, about 2 equivalents to about 80 equivalents, about 2 equivalents to about 60 equivalents, about 2 equivalents to about 50 equivalents, about 2 equivalents to about 40 equivalents, about 2 equivalents to about 30 equivalents, about 2 equivalents to about 20 equivalents, about 2 equivalents to about 10 equivalents, about 2 equivalents to about 8 equivalents, about 2 equivalents to about 6 equivalents, or about 4 equivalents to about 6 equivalents.

In some embodiments, the co-enzyme regeneration conditions comprises NADH at about 0.05 equivalents to about 5 equivalents relative to a 2′, 3′ and/or 5′ substituted N-heteroaryl or a derivative thereof concentration. In other embodiments, the NADH concentration is about 0.1 equivalents to about 5 equivalents, about 0.5 equivalents to about 5 equivalents, about 1 equivalents to about 5 equivalents, about 1.5 equivalents to about 5 equivalents, about 2 equivalents to about 5 equivalents, about 2.5 equivalents to about 5 equivalents, about 3 equivalents to about 5 equivalents, or about 4 equivalents to about 5 equivalents.

In some embodiments, the co-enzyme regeneration conditions comprises monosaccharide at about 5 equivalents to about 500 equivalents relative to a flavin-dependent halogenase concentration. In other embodiments, the concentration is about equivalents to about 450 equivalents, about 5 equivalents to about 400 equivalents, about 5 equivalents to about 350 equivalents, about 5 equivalents to about 300 equivalents, about 5 equivalents to about 250 equivalents, about 5 equivalents to about 200 equivalents, about 5 equivalents to about 150 equivalents, about 5 equivalents to about 100 equivalents, about 5 equivalents to about 80 equivalents, about 5 equivalents to about 60 equivalents, about 5 equivalents to about 50 equivalents, about 5 equivalents to about 40 equivalents, about 5 equivalents to about 30 equivalents, about 5 equivalents to about 20 equivalents, or about 5 equivalents to about 10 equivalents.

In some embodiments, the co-enzyme regeneration conditions comprises monosaccharide at about 5 equivalents to about 20 equivalents relative to a 2′, 3′ and/or 5′ substituted N-heteroaryl or a derivative thereof concentration. The monosaccharide may be glucose (dextrose), fructose (levulose), or galactose. In some embodiments, the monosaccharide concentration is about 8 equivalents to about 20 equivalents, about 8 equivalents to about 18 equivalents, about 8 equivalents to about 16 equivalents, about 8 equivalents to about 14 equivalents, about 8 equivalents to about 12 equivalents, or about 10 equivalents to about 12 equivalents.

In some embodiments, the co-enzyme regeneration conditions comprises flavin adenine dinucleotide (FAD) at about 0.001 equivalents to about 0.2 equivalents relative to a flavin-dependent halogenase concentration. In other embodiments, the concentration is about 0.02 equivalents to about 0.2 equivalents, about 0.04 equivalents to about 0.2 equivalents, about 0.06 equivalents to about 0.2 equivalents, about 0.08 equivalents to about 0.2 equivalents, about 0.1 equivalents to about 0.2 equivalents, about 0.12 equivalents to about 0.2 equivalents, about 0.14 equivalents to about 0.2 equivalents, about 0.16 equivalents to about 0.2 equivalents, or about 0.18 equivalents to about 0.2 equivalents. In other embodiments, the concentration is about 0.001 equivalents to about 0.18 equivalents, about 0.001 equivalents to about 0.16 equivalents, about 0.001 equivalents to about 0.14 equivalents, about 0.001 equivalents to about 0.12 equivalents, about 0.001 equivalents to about 0.1 equivalents, about 0.001 equivalents to about 0.05 equivalents, about 0.001 equivalents to about 0.01 equivalents, about 0.001 equivalents to about 0.008 equivalents, about 0.001 equivalents to about 0.006 equivalents, about 0.001 equivalents to about 0.005 equivalents, about 0.001 equivalents to about 0.004 equivalents, or about 0.001 equivalents to about 0.003 equivalents.

In some embodiments, the co-enzyme regeneration conditions comprises flavin adenine dinucleotide (FAD) at about 0.1 mol % to about 1 mol % relative to a 2′, 3′ and/or 5′ substituted N-heteroaryl or a derivative thereof concentration. In other embodiments, the concentration is about 0.1 mol % to about 0.8 mol %, about 0.1 mol % to about 0.6 mol %, about 0.1 mol % to about 0.5 mol %, about 0.1 mol % to about 0.4 mol %, or about 0.1 mol % to about 0.3 mol %. In some embodiments, the concentration is about 0.2 mol %.

In some embodiments, the co-enzyme regeneration conditions comprises E. coli flavin reductase (Fre) at about 0.001 equivalents to about 0.5 equivalents relative to a flavin-dependent halogenase concentration. In other embodiments, the concentration is about 0.2 equivalents to about 0.5 equivalents, about 0.3 equivalents to about 0.5 equivalents, or about 0.4 equivalents to about 0.5 equivalents. In other embodiments, the concentration is about 0.001 equivalents to about 0.1 equivalents, about 0.001 equivalents to about 0.05 equivalents, about 0.001 equivalents to about 0.01 equivalents, about 0.001 equivalents to about 0.008 equivalents, about 0.004 equivalents to about 0.008 equivalents, about 0.004 equivalents to about 0.006 equivalents. In other embodiments, the concentration is about 0.005 equivalents.

In some embodiments, the co-enzyme regeneration conditions comprises E. coli flavin reductase (Fre) at about 0.1 mol % to about 2 mol % relative to a 2′, 3′ and/or 5′ substituted N-heteroaryl or a derivative thereof concentration. In other embodiments, the concentration is about 0.2 mol % to about 2 mol %, about 0.4 mol % to about 2 mol %, about 0.5 mol % to about 2 mol %, about 0.5 mol % to about 1.8 mol %, about 0.5 mol % to about 1.6 mol %, about 0.5 mol % to about 1.4 mol %, about 0.5 mol % to about 1.2 mol %, or about 0.5 mol % to about 1 mol %. In other embodiments, the concentration is about 0.5 mol %.

The method may comprise a co-factor regeneration system using glucose dehydrogenase (GdHi) to regulate a more consistent supply of NADH over time, enabling high conversion as compared to system without the use of GDH.

In some embodiments, the co-enzyme regeneration conditions comprises glucose 1-dehydrogenase (GdHi) at about 0.001 equivalents to about 0.5 equivalents relative to a flavin-dependent halogenase concentration. In other embodiments, the concentration is about 0.2 equivalents to about 0.5 equivalents, about 0.3 equivalents to about 0.5 equivalents, or about 0.4 equivalents to about 0.5 equivalents. In other embodiments, the concentration is about 0.001 equivalents to about 0.1 equivalents, about 0.001 equivalents to about 0.05 equivalents, about 0.001 equivalents to about 0.01 equivalents, about 0.001 equivalents to about 0.008 equivalents, about 0.004 equivalents to about 0.008 equivalents, about 0.004 equivalents to about 0.006 equivalents. In other embodiments, the concentration is about 0.005 equivalents.

In some embodiments, the co-enzyme regeneration conditions comprises glucose dehydrogenase (GdHi) at about 0.1 mol % to about 2 mol % relative to a 2′, 3′ and/or 5′ substituted N-heteroaryl or a derivative thereof concentration. In other embodiments, the concentration is about 0.2 mol % to about 2 mol %, about 0.4 mol % to about 2 mol %, about 0.5 mol % to about 2 mol %, about 0.5 mol % to about 1.8 mol %, about 0.5 mol % to about 1.6 mol %, about 0.5 mol % to about 1.4 mol %, about 0.5 mol % to about 1.2 mol %, or about 0.5 mol % to about 1 mol %. In other embodiments, the concentration is about 0.5 mol %.

In some embodiments, a ratio of flavin adenine dinucleotide (FAD): E. coli flavin reductase (Fre):glucose dehydrogenase (GDH) is about 1:2.5:2.5.

In some embodiments, a ratio of FAD:Fre:NADH:GDH is about 1:2.5:2500:2.5.

In some embodiments, the co-enzyme regeneration conditions further comprises a halogen at about 10 equivalents to about 30 equivalents relative to a 2′, 3′ and/or 5′ substituted N-heteroaryl or a derivative thereof concentration. In other embodiments, the concentration is about 10 equivalents to about 25 equivalents, about 10 equivalents to about 20 equivalents, about 10 equivalents to about 15 equivalents, or about 10 equivalents to about 12 equivalents.

In some embodiments, the halogen is derived from a halide, the halide selected from Cl⁻ or Br⁻. For example an inorganic halide salt. The inorganic halide salt may be MgBr₂ or MgCl₂. Sodium and potassium salts may also be used.

In some embodiments, the co-enzyme regeneration conditions further comprises a buffer. The buffer may be phosphate buffer or Tris HCl buffer. The concentration of the buffer can be from about 10 mM to about 50 mM.

In some embodiments, flavin-dependent halogenases (or PrnC biocatalyst) is at about 2 mol % to about 10 mol % relative to a 2′, 3′ and/or 5′ substituted N-heteroaryl or a derivative thereof concentration. In other embodiments, the concentration is about 2 mol % to about 8 mol %, about 2 mol % to about 6 mol %, or about 2 mol % to about 4 mol %.

In some embodiments, the flavin-dependent halogenases is monodechloroaminopyrrolnitrin halogenase (PrnC). In other embodiments, the enzyme is selected from halB and tryptophan 7-halogenase (RebH). halB is a halogenase gene isolated from a cosmid library of the pentachloropseudilin producer Actinoplanes sp. ATCC 33002.

In order to further improve the yield of the pyrrole product, the inventors also explored the overexpression of the Flavin-dependent halogenase gene. Halogenating activity can only be detected after the halogenase gene is highly overexpressed and soluble halogenase is produced. Initial attempts at heterologous expression of PrnC yielded <1 mg/L as the majority of the protein were insoluble. After subsequent optimization of the expression construct, we discovered that addition of a N-terminal 11 amino acid solubility tag (NT11), together with a C-terminal His₆-tag (6His), allowed for the production of sufficient quantities of soluble functional PrnC, and purification by metal affinity chromatography (~9 mg/L culture).

Specifically, engineering of a NT11 soluble tag into the PrnC gene enabled the improvement in heterologous expression yield of the PrnC in E. coli by at least 18-fold, whilst keeping the protein functional. It is believed that the tag allowed the protein to fold better and thus perform more consistently.

Accordingly, in some embodiments, the flavin-dependent halogenases comprises a N-terminal 11 amino acid solubility tag. In some embodiments, the flavin-dependent halogenases comprises a N-terminal 11 amino acid solubility tag and a C-terminal His6-tag. The His6 tag may be used for affinity purification.

In some embodiments, the N-terminal 11 amino acid solubility tag is derived from a first 11 amino acid residues within a N-terminal N-half domain of a duplicated carbonic anhydrase (dCA) from Dunaliella species. For example, the N-terminal 11 amino acid solubility tag may be as described in Nguyen, Thi Khoa My, et al. Applied microbiology and biotechnology 103.5 (2019): 2205-2216), the reference of which is incorporated herein.

In some embodiments, the 11 amino acid solubility tag has an amino acid sequence of VSEPHDYNYEK. In some embodiments, the solubility tag is selected from Human influenza hemagglutinin (HA) tag, Small Ubiquitin-like Modifier (SUMO), maltose-binding protein (MBP) and Glutathione-S-transferase (GST) tag. The HA-tag is derived from the HA-molecule corresponding to amino acids 98-106.

In some embodiments, the method was performed for at least 4 h. In other embodiments, the method was performed for at least 6 h, 8 h, 10 h, 12 h, 14 h, 16 h or 18 h.

In some embodiments, the method was performed at a temperature of about 25° C. to about 45° C. In other embodiments, the temperature is about 30° C.

In some embodiments, method was performed under constant mixing. The mixing may be by orbital shaking at about 300 rpm.

In some embodiments, the compound of Formula (I) is selected from:

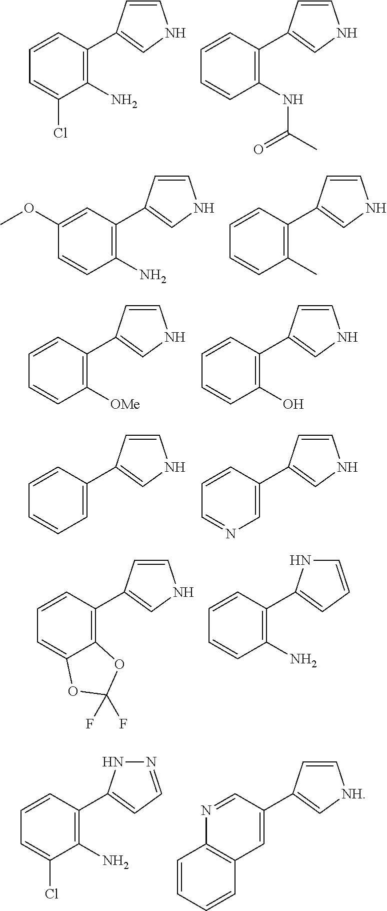

In some embodiments, the compound of Formula (I) is selected from:

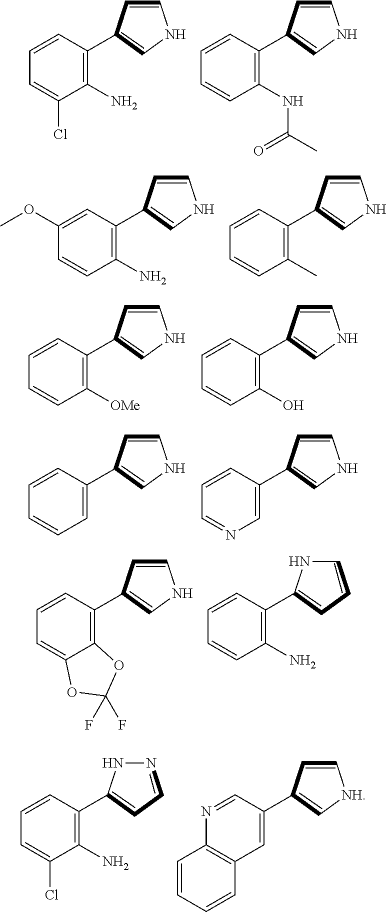

In some embodiments, the halogenated compound of Formula (I) is (where X₁ represents halo):

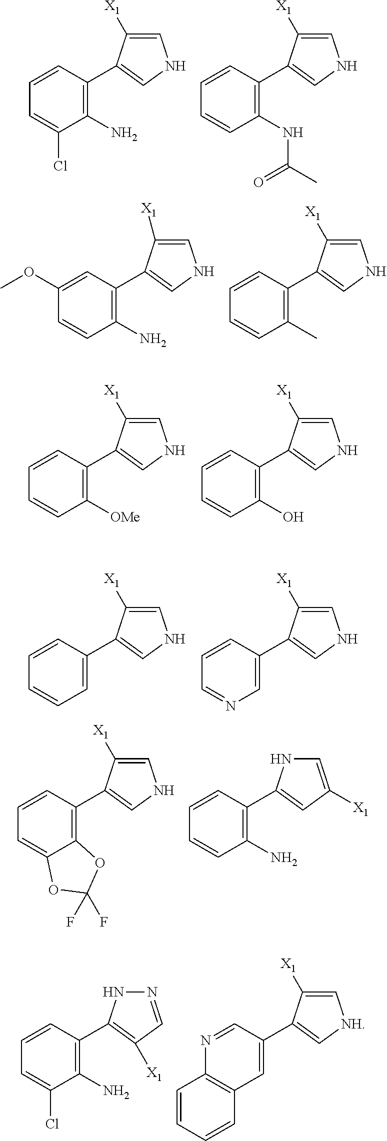

In some embodiments, the halogenated compound of Formula (I) is (where X₁ represents halo):

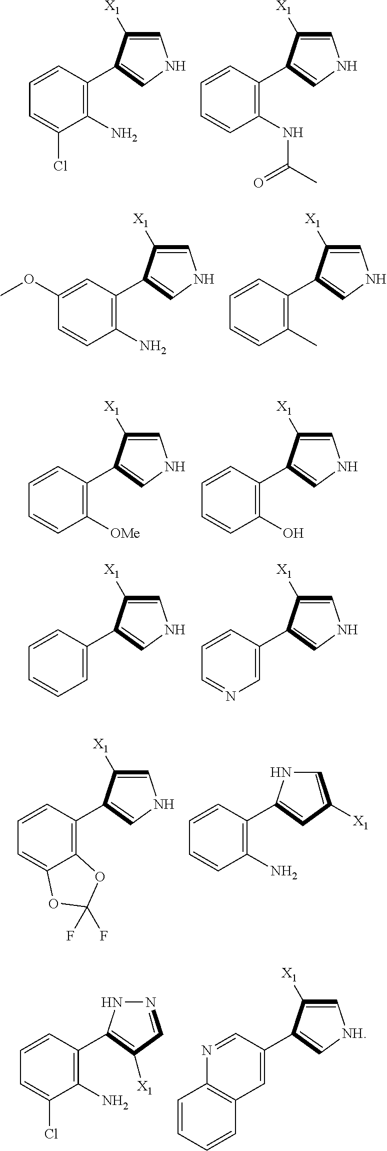

In some embodiments, the method further comprises a step of purifying the halogenated compound of Formula (I).

In some embodiments, the method is characterised by a K_{cat} of about 7×10⁻³ s⁻¹ to about 8×10⁻³ s⁻¹.

In some embodiments, the method is characterised by a Kₘ of about 1×10⁻⁵ M to about 2×10⁻⁵ M.

In some embodiments, the method is characterised by a K_{cat}/Kₘ of about 4×10² s⁻¹ M⁻¹ to about 5×10² s⁻¹ M⁻¹.

The present disclosure also concerns a flavin-dependent halogenase and its clonal construct thereof, comprising:
a) a 11 amino acid solubility tag at a N-terminus of the flavin-dependent halogenase; and
b) a His6 tag at a C-terminus of the flavin-dependent halogenase.

The present disclosure also concerns a method of producing a flavin-dependent halogenase, comprising:
a) tagging a 11 amino acid solubility tag at a N-terminus of the flavin-dependent halogenase; and
b) tagging a His6 tag at a C-terminus of the flavin-dependent halogenase.

In some embodiments, the flavin-dependent halogenase is expressed in a cell. The cell may be a bacterium, such as E. coli.

Accordingly, the method comprises expressing the flavin-dependent halogenase in a cell, wherein the flavin-dependent halogenase comprises an 11 amino acid solubility tag at a N-terminus thereof and a His6 tag at a C-terminus thereof.

In some embodiments, the method further comprises a step of purifying the flavin-dependent halogenase by metal affinity chromatography. In some embodiments, the method further comprises a step of purifying the flavin-dependent halogenase from the cell. The purification step may involve binding the enzyme to a resin and eluting the enzyme from the resin.

The present disclosure also concerns a method of synthesising Fludioxonil, comprising: a) contacting a compound of formula (II) or derivative thereof with a halogen and a flavin-dependent halogenase under co-enzyme regeneration conditions in order for the halogen to be substituted at a 4′ position on the compound or derivative thereof;

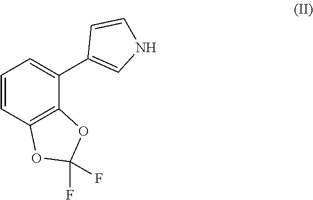 [SMILES: FC1(F)Oc2cccc(-c3cc[nH]c3)c2O1]

wherein the co-enzyme regeneration conditions comprises flavin adenine dinucleotide (FAD) at about 0.01 equivalents to about 0.2 equivalents relative to a flavin-dependent halogenase concentration;
E. coli flavin reductase (Fre) at about 0.1 equivalents to about 0.5 equivalents relative to a flavin-dependent halogenase concentration;
nicotinamide adenine dinucleotide hydrogen (NADH) at about 2 equivalents to about 250 equivalents relative to a flavin-dependent halogenase concentration;
glucose 1-dehydrogenase (GdHi) at about 0.1 equivalents to about 0.5 equivalents relative to a flavin-dependent halogenase concentration;
monosaccharide at about 5 equivalents to about 500 equivalents relative to a flavin-dependent halogenase concentration;
b) contacting the 4′ halogenated compound of step a) with a palladium precatalyst and a cyanide precursor.

### Examples

Halogenase cloning, expression and purification. NT11¹-PrnC-6His construct in pET-28a(+) was ordered from Twist Biosciences as a clonal construct and transformed into T7 Express E. coli (NEB). The resulting strain was cultured in 1 L LB media at 37° C. When OD₆₀₀ reached 0.4, 0.1 mM IPTG was used to induce for overnight expression at 16° C. After expression, the cultures were centrifuged at 10,000 g for 10 minutes at 4° C. The resulting pellets were resuspended in 20 mL of 100 mM sodium phosphate pH 7, 10 mM imidazole, 150 mM sodium chloride before sonication. After sonication, the resulting lysate was then centrifuged at 19,000 g for 1 hour at 4° C. The supernatant was incubated with Ni-NTA agarose for 1 hour at 4° C. The resin was washed with 20 mL 100 mM sodium phosphate pH 7-7.4, 80 mM imidazole, 150 mM sodium chloride, 10% glycerol and the bound protein was eluted with 5 mL of 100 mM sodium phosphate pH 7-7.4, 150-500 mM imidazole, 50 mM sodium chloride, 10% glycerol. The elution was buffer exchanged and concentrated with 50 mM sodium phosphate pH 7-7.4, 10% glycerol. The final yields of NT11-PrnC were an estimated 9 mg/L culture. In comparison, PrnC alone (or with various placements of his-tags) were expressed at yields of <0.5 mg/L culture and give inconsistent assay results.

Glucose dehydrogenase (GdHi or GDH). Purchased from Sigma-aldrich with activity units ≥200 U/mg.

Method for Fre cloning, expression and purification: The nucleic acid sequence that encodes for Flavin reductase Fre was purchased as a gBlock from Integrated DNA Technologies. Fre sequence was cloned into pET-28a(+) vector via NEBuilder® HiFi DNA Assembly method and transformed into E. coli Acella (EdgeBio). E. Coli strain expressing Fre was cultured in 1 L of LB Kan⁵⁰ media at 37° C. At OD₆₀₀ 0.4-0.6, 0.1 mM IPTG was used to induce protein expression at 16° C. over 18 h. Cell culture was harvested by centrifugation at 4000 rcf for 10 min at 4° C. After media was decanted, cell pellet was resuspended in 30 ml of 50 mM tris pH 7.4, 300 mM sodium chloride, 10 mM imidazole and lysed by cell disruption. Cell lysate was centrifuged at 33,600 rcf for 45 min at 4° C. to differentiate supernatant from insoluble debris. Fre proteins from lysate supernatant were purified using immobilized metal affinity chromatography via TALON resins interaction with N-terminus His-tag Fre. After lysate supernatant was applied, 10 ml of 50 mM tris pH 7.4, 300 mM sodium chloride, 10 mM imidazole was used to wash the resins. Fre proteins were eluted from the resins using 5 ml of 50 mM tris pH 7.4, 300 mM sodium chloride, 200 mM imidazole. Eluted samples were buffer exchanged and concentrated with 50 mM tris pH 7.4, 100 mM NaCl, 10% glycerol

Method for PrnC mutants construction in mutagenesis studies. Single-site mutations of the prnC gene were constructed via overlap extension PCR, using PrimeSTAR Max DNA Polymerase, on the pET-28a(+) NT11-PrnC-6His plasmid template. The mutagenesis primers used are as shown:

E60A forward primer
(SEQ ID NO: 1)
5′-CGAAAGTTCCATCCCGGCGACTTCGTTGATGAATC-3′;
E60A reverse primer
(SEQ ID NO: 2)
5′-GATTCATCAACGAAGTCGCCGGGATGGAACTTTCG-3′;
K97A forward primer
(SEQ ID NO: 3)
5′-CATCGTCAACCGGAATTGCGCGTAATTTCGGCTTTG-3′;
K97A reverse primer
(SEQ ID NO: 4)
5′-CAAAGCCGAAATTACGCGCAATTCCGGTTGACGATG-3′;
E129A forward primer
(SEQ ID NO: 5)
5′-GCTTCCCTGGGGACCTGCGTCACATTATTATCGTC-3′;
E129A reverse primer
(SEQ ID NO: 6)
5′-GACGATAATAATGTGACGCAGGTCCCCAGGGAAGC-3′.

Standard flanking primers for prnC gene were used for amplification of the respective PCR segments in each mutant construct:

prnC forward primer
(SEQ ID NO: 7)
5′-GGAGATATACCATGGTAAGTGAACCCCACGACTATAATTATG-3′;
prnC reverse primer
(SEQ ID NO: 8)
5′-GTGGTGGTGCTCGAGTTTTTTCAGCGCTAATCCAATACGC-3′.

PCR segments and linearized plasmids were ligated using NEBuilder Hifi DNA assembly protocol and the mutations were confirmed by DNA sequencing.

Sequences - NT11-PrnC - nt
(SEQ ID NO: 9)
ATGGTAAGTGAACCCCACGACTATAATTATGAGAAAGCTAGCGCG
TCTATGACACAAAAATCTCCCGCCAACGAGCACGATTCAAATCAC
TTTGATGTCATTATTCTGGGATCGGGCATGTCAGGTACGCAGATG
GGCGCTATTTTGGCTAAACAACAATTTCGTGTGTTAATTATTGAA
GAGTCCAGCCACCCACGTTTTACCATTGGCGAAAGTTCCATCCCG
GAAACTTCGTTGATGAATCGTATCATCGCAGACCGCTATGGTATC
CCAGAACTGGACCATATTACCTCCTTTTATAGCACACAACGTTAC
GTCGCATCGTCAACCGGAATTAAACGTAATTTCGGCTTTGTATTT
CACAAACCAGGTCAAGAGCACGATCCTAAGGAATTCACACAGTGT
GTGATTCCGGAGCTTCCCTGGGGACCTGAATCACATTATTATCGT
CAGGACGTAGATGCATATTTATTGCAGGCGGCGATCAAGTACGGG
TGTAAAGTGCACCAAAAGACAACTGTGACGGAGTATCATGCTGAT
AAGGATGGCGTAGCGGTCACAACAGCTCAAGGGGAACGCTTCACA
GGTCGCTACATGATCGATTGCGGTGGACCCCGCGCGCCCTTGGCT
ACTAAATTCAAGTTACGTGAAGAGCCATGCCGTTTTAAAACGCAC
AGCCGTTCTCTGTATACTCACATGTTAGGGGTCAAGCCATTCGAC
GACATTTTTAAAGTAAAGGGGCAGCGCTGGCGTTGGCATGAAGGA
ACCCTTCATCACATGTTCGAAGGAGGATGGCTGTGGGTGATTCCC
TTTAATAACCATCCCCGTAGCACAAATAACTTAGTCTCGGTTGGT
TTGCAACTTGACCCACGTGTCTACCCCAAGACCGACATCTCCGCT
CAACAAGAATTCGACGAGTTCCTTGCCCGTTTTCCGTCAATCGGA
GCCCAGTTTCGCGACGCCGTTCCAGTTCGCGATTGGGTGAAAACT
GATCGCCTTCAATTTTCTAGTAATGCATGCGTCGGAGACCGTTAC
TGTTTAATGTTACACGCGAACGGATTTATTGATCCGTTGTTTTCG
CGTGGGCTTGAGAATACTGCGGTCACGATCCACGCCTTAGCTGCG
CGTCTTATCAAGGCCCTGCGCGACGATGACTTCTCTCCAGAACGC
TTCGAGTATATCGAGCGTTTGCAACAGAAGTTGTTGGACCACAAT
GACGACTTCGTGTCATGTTGCTACACAGCTTTTTCGGATTTTCGT
CTTTGGGATGCCTTTCACCGCCTTTGGGCCGTGGGGACTATCCTT
GGACAATTTCGTCTTGTGCAGGCCCACGCACGCTTTCGCGCATCG
CGTAACGAGGGTGATCTGGATCATTTAGATAACGACCCACCCTAT
TTGGGGTATCTGTGCGCTGATATGGAAGAGTACTATCAGCTTTTC
AATGACGCCAAGGCTGAGGTGGAAGCAGTATCAGCGGGACGTAAG
CCTGCCGACGAGGCTGCCGCGCGCATTCATGCCTTGATCGACGAA
CGTGACTTCGCCAAACCTATGTTCGGGTTCGGTTACTGCATTACA
GGAGATAAACCACAGTTGAATAATTCCAAGTACTCTTTATTGCCT
GCGATGCGCTTGATGTACTGGACGCAGACCCGTGCCCCAGCGGAA
GTCAAGAAGTATTTCGATTATAACCCTATGTTTGCGTTGCTTAAG
GCATATATTACTACGCGTATTGGATTAGCGCTGAAAAAACTCGAG
CACCACCACCACCACCACTGA
Sequences - NT11-PrnC - aa
(SEQ ID NO: 10)
MVSEPHDYNYEKASASMTQKSPANEHDSNHFDVIILGSGMSGTQM
GAILAKQQFRVLIIEESSHPRFTIGESSIPETSLMNRIIADRYGI
PELDHITSFYSTORYVASSTGIKRNFGFVFHKPGQEHDPKEFTQC
VIPELPWGPESHYYRQDVDAYLLQAAIKYGCKVHQKTTVTEYHAD
KDGVAVTTAQGERFTGRYMIDCGGPRAPLATKFKLREEPCRFKTH
SRSLYTHMLGVKPFDDIFKVKGQRWRWHEGTLHHMFEGGWLWVIP
FNNHPRSTNNLVSVGLQLDPRVYPKTDISAQQEFDEFLARFPSIG
AQFRDAVPVRDWVKTDRLQFSSNACVGDRYCLMLHANGFIDPLFS
RGLENTAVTIHALAARLIKALRDDDFSPERFEYIERLQQKLLDHN
DDFVSCCYTAFSDFRLWDAFHRLWAVGTILGQFRLVQAHARFRAS
RNEGDLDHLDNDPPYLGYLCADMEEYYQLFNDAKAEVEAVSAGRK
PADEAAARIHALIDERDFAKPMFGFGYCITGDKPQLNNSKYSLLP
AMRLMYWTQTRAPAEVKKYFDYNPMFALLKAYITTRIGLALKKLE
HHHHHH

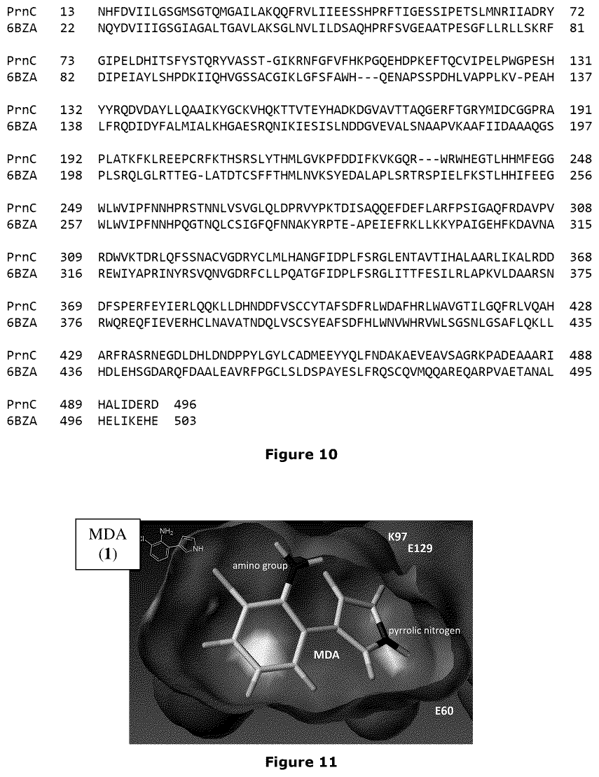

Homology modelling and substrate docking of NT11-PrnC. Homology modelling was performed in Modeller v10 program and generated 100,000 homology models based on the template crystal structure of halogenase PItM (PDB code: 6BZA) whose sequence was aligned with that of PrnC (FIG. 10) with the sequence identity of 36%. Subsequently, all the models were subjected to the backbone, sidechain and loop optimization respectively, and then the top 50 optimized models with the lowest DOPE scores were used for the subsequent molecular docking with the native substrate (1) by using GOLD v2018 program with the optimal docking parameters (the binding pocket is defined by the ligand copied from the crystal structure of 6BZA with the spherical radius of 8.0 Angstrom; scoring function is GoldScore; population size is 500; the number of operations is 500,000; number of island is 10; crossover frequency is 95%; mutation frequency is 95%; migration frequency is 20%; the number of output docking solutions is 3), which afforded 150 docking solutions in total.

In order to achieve an optimal model for the PrnC/1 complex, 150 docking solutions were subjected to the further inspection based on the following two criteria: (1) there must be a nearby lysine residue stretching towards the pyrrole ring in 1, because a lysine is required for the catalysis; (2) the hydrogen on the pyrrolic nitrogen of 1 should form a hydrogen bond with the hydrogen acceptor of a residue to stabilize the intermediate during the catalytic reaction. After the manual and visual examination, a reasonable model of PrnC/1 complex, which satisfied both criteria above, was harvested and shown in FIG. 2 and FIG. 11.

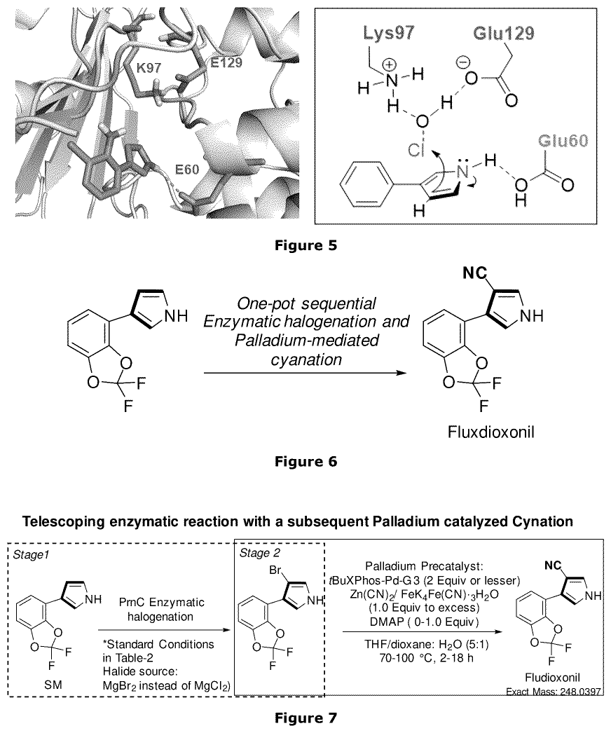

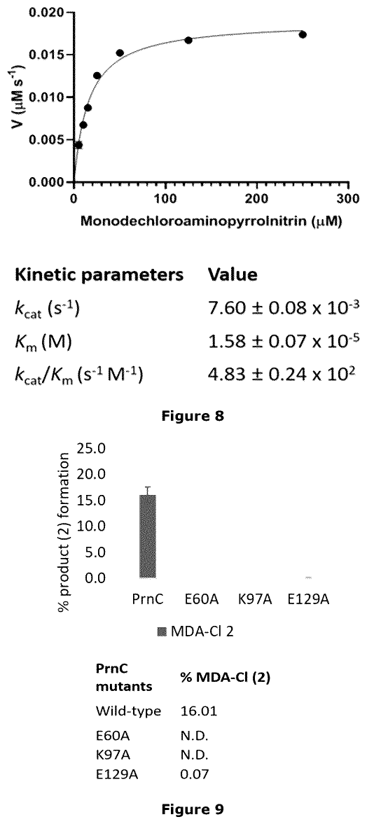

To determine which residues are important to the catalytic reaction of PrnC enzyme, we conducted a systematic computational modeling of the enzyme and constructed a three-dimensional model for the PrnC/substrate complex of PrnC (FIG. 8). The PrnC/substrate model (FIG. 5) indicates that the lysine residue K97 is close to 1 and may result in the initiation of catalysis; E129 forms a salt bridge with K97 to stabilize its sidechain conformation and E60 may form a hydrogen bond with 1. To further validate the importance of our computationally proposed key residues, an experimental mutagenesis study was conducted, and its results are shown in FIG. 9. The mutations K97 and E60 completely abolished enzyme activity, indicating that K97 is a key catalytic residue with E60 playing a critical role in substrate stabilization by H-bonding. As a consequence of the E129A mutation the enzyme activity was significantly reduced, revealing a preference for a spatial orientation of the K97 residue. It appears that the catalytic Glu residue E129 plays a key role in improving HOX's electrophilicity by interacting with K97 (FIG. 9) or serving as a general base to deprotonate a Wheland-type pyrrolic intermediate. These mutagenesis data show that our concept is plausible from this point of view.

### Analytical Scale Biotransformations:

NT-11 PrnC-Catalyzed halogenation. In a solution containing the pyrrolic derivative starting material (0.5 mM), MgCl₂/MgBr₂ (10 mM), glucose (5.0 mM), FAD (1.0 M), NT-11 PrnC (12.5 μM), Fre (2.5 M) and Gdhi (2.5 μM) in 10 mM potassium phosphate buffer, NADH (2.5 mM) was added to a total volume of 200 μL. After an overnight incubation of 30° C. and orbital shaking at 350 rpm, reactions were quenched with an equivalent volume of MeOH, pelleted by centrifugation (15000 rpm for 10 min) and the supernatant analyzed by HPLC-MS using the analytical HPLC method.

Determination of Kinetic Parameters for NT-11 PrnC and PrnC mutant assay. Kinetic analysis of PrnC (2.5 μM) activity against MDA was performed over a 5-250 μM substrate concentration range. The assay reaction was supplemented with Fre (2.5 μM), FAD (1 μM) and MgCl₂ (10 mM) in 20 mM Tris buffer, pH 7.4. NADH (2.5 mM) was added last for reaction initiation. The amount of products formed were measured at 120, 300 and 600 seconds via a Kinetex XB-C18 reversed-phased column (2.6 μm, 150×4.6 mm) on a Shimadzu LC-20AD HPLC. Absorbance at λ=254 nm was used to monitor product formation during an isocratic flow rate of 0.6 mL/min (50% MeCN/H₂O+0.1% TFA) over 10 min. Kinetic parameters were determined by nonlinear fitting of a Michaelis-Menten curve using the GraphPad Prism software. Activity assays for PrnC mutants were performed in lysates with the respective over-expressed protein variant. Wild-type PrnC enzyme was used as a positive control and reaction conditions were similar to the 18 h assay method described above. The amount of products formed by PrnC mutants were quantified using the analytical HPLC method.

### Preparative Scale Cell Lysate Biotransformations

### Method for PrnC Lysate Preparation

E. Coli strain expressing PrnC was cultured in 1 L of LB Kan⁵⁰ media at 37° C. At OD₆₀₀ 0.4-0.6, 0.1 mM IPTG was used to induce protein expression at 16° C. over 18 h. Cell culture was harvested by centrifugation at 4000 rcf for 10 min at 4° C. After media was decanted, cell pellet was resuspended in 30 ml of 50 mM tris pH 7.4, 300 mM sodium chloride, 10 mM imidazole and lysed by cell disruption. Cell lysate was centrifuged at 33,600 rcf for 45 min at 4° C. to differentiate supernatant from insoluble debris. Lysate supernatant was buffer exchanged into 50 mM tris pH 7.4 in preparation for activity assay.

PrnC-Cell lysate Chlorination. In a solution of the PrnC cell lysate (0.01 mM, enzyme loading ~2.0 mol %, total volume 20 mL), the pyrrolic derivative starting material (0.5 mM), MgCl₂ (10 mM), glucose (5.0 mM), FAD (1.0 μM), Fre (2.5 μM), GDH2 (2.5 μM), NADH (2.5 mM) was added and allowed to stir at 30° C. with a stir bar in a petri dish at 30 rpm.

After an overnight incubation of 25° C. and stir-bar shaking at 30 rpm, reactions were quenched with an equivalent volume of (1:1) MeOH-brine solution. The aqueous layer was 3× extracted with 20 ml of ethyl acetate pelleted by centrifugation (40 rpm for 10 min). The combined organic layers were dried over Na₂SO₄, filtered, and concentrated before purification by semi-preparative HPLC.

### General HPLC and LC-MS Methods

Analytical methods: Spectroscopic grade solvents were purchased from Sigma Aldrich. Low-resolution LC-MS spectra were recorded on an Agilent LCMS machine with dual MM-APCI-ES. High-resolution mass spectra (HRMS) were recorded on an Agilent ESI-TOF mass spectrometer at 3500 V emitter voltage. Exact m/z values are reported in Daltons.

Semi-Preparative HPLC method. 900 μL of the crude mixture dissolved in H₂O/MeCN was injected onto a Phenomenex Jupiter® semi-preparative C18 HPLC column (90 Å, 5 μm packing, 250×10 mm) and purified using reverse phase chromatography. Gradient starting conditions of 5% MeCN/H₂O (+0.1% Formic acid) to 25% MeCN/H₂O over 10 min, followed by 25% MeCN/H₂O into 50% MeCN/H₂O over 20 min, followed by 50% MeCN/H₂O into 75% MeCN/H₂O over 10 min, followed by 75% MeCN/H₂O into 95% MeCN/H₂O over 5 min, followed by a hold at 95% MeCN/H₂O for 5 min. Column condition was equilibrated back to starting conditions over 2 mins post-run. Flow rates were kept constant at 3 mL/min. UV absorbance was monitored at 220 nm, 254 nm and 280 nm.

Analytical HPLC Method. 10 μL of the supernatant injected onto SecurityGuard™ column (KJ0-4282) with a (4.0 mm×3.0 mm) guard cartridge before separation using a Phenomenex Gemini® C18 analytical column (5 μm packing, 150 mm×4.6 mm). Gradient starting conditions of 5% MeCN/H₂O (+0.1% Formic acid) were held for 1 min before development into 50% MeCN/H₂O over 3 min, followed by development into 95% MeCN/H₂O over 3 min. 95% MeCN/H₂O was held for 1 min before equilibration back to starting conditions over 1 min. Starting conditions was held for 1 min followed by another 2 min post-run. Flow rates were kept constant at 1 mL/min. Column temperature was kept constant at 30° C. UV absorbance was detected at 220 nm, 254 nm and 210 nm throughout the run.

General LC-MS Method. 10 μL of the supernatant was separated using the appropriate analytical HPLC method described above. Detection was performed using an Agilent® single quadrupole LC/MSD system.

### Synthesis of Substrates & Standards

A substrate scope of the enzyme by the application of the Prnc enzyme on a list of structural diverse pyrrolic substrates is determined. An optimized co-factors regeneration protocol was used to regulate the generation of the activated halogen which is captured by the Prnc enzyme into the active site for the bioconversion. Reaction is conducted at ambient temperature of 30° C., in an aqueous non-toxic buffer solution with an environmentally friendly chloride source.

TABLE 1
Optimization.

| | | | No catalase | | NADH | | Tris buffer | | Fre | |
|---|---|---|---|---|---|---|---|---|---|---|
| Reaction component | 1 | | 2 | | 3 | | 4 | | 5 | |
| FAD (μM) | 1 | 1 | 1 | 1 | 1 | 1 | 1 | 1 | 1 | 1 |
| NADH (mM) | 2.5 | 2.5 | 2.5 | 2.5 | 25 μM | 25 μM | 2.5 | 2.5 | 2.5 | 2.5 |
| MgCl₂ (mM) | 10 | 10 | 10 | 10 | 10 | 10 | 10 | 10 | 10 | 10 |
| PrnC substrate (mM) | 0.5 | 0.5 | 0.5 | 0.5 | 0.5 | 0.5 | 0.5 | 0.5 | 0.5 | 0.5 |
| Catalase (U/ml) | 33 | 33 | — | — | 33 | 33 | 33 | 33 | 33 | 33 |
| Glucose (μM) | 5 | 5 | 5 | 5 | 5 | 5 | 5 | 5 | 5 | 5 |
| GDH (μM) | 2.5 | 2.5 | 2.5 | 2.5 | 2.5 | 2.5 | 2.5 | 2.5 | 2.5 | 2.5 |
| Fre (μM) | 2.5 | 2.5 | 2.5 | 2.5 | 2.5 | 2.5 | 2.5 | 2.5 | 5 | 5 |
| Prnc (μM) | 10 | — | 10 | — | 10 | — | 10 | — | 10 | — |

| 10 mM sodium | Top up to 200 μL | — | Top up to |
|---|---|---|---|
| phosphate pH 7.0 | | | 200 μL |

| 50 mM Tris pH 7.4 | — | Top up to | — |
|---|---|---|---|
| | | 200 μL | |

TABLE 2
Optimization.

| Reaction | | 5x NadH, | 0.05 | No | Std |
|---|---|---|---|---|---|
| component | Iodide | No Gdhi | NADH | PrnC | Conditions |
| FAD (μM) | 1 | 1 | 1 | 1 | 1 |
| NADH (mM) | 2.5 | 2.5 | 0.05 | 2.5 | 2.5 |
| MgCl₂ (mM) | — | 10 | 10 | 10 | 10 |
| PrnC | 0.5 | 0.5 | 0.5 | 0.5 | 0.5 |
| substrate (mM) | | | | | |
| Iodide (mm) | 20 | — | — | — | — |
| Glucose (mM) | 5 | — | 5 | 5 | 5 |
| GDH (μM) | 2.5 | — | 2.5 | 2.5 | 2.5 |
| Fre (μM) | 2.5 | 2.5 | 2.5 | 2.5 | 2.5 |
| Prnc (μM) | 12.5 | 12.5 | 12.5 | 0 | 12.5 |

TABLE 3
Optimization study of PrnC biocatalyst on the native substrate 1

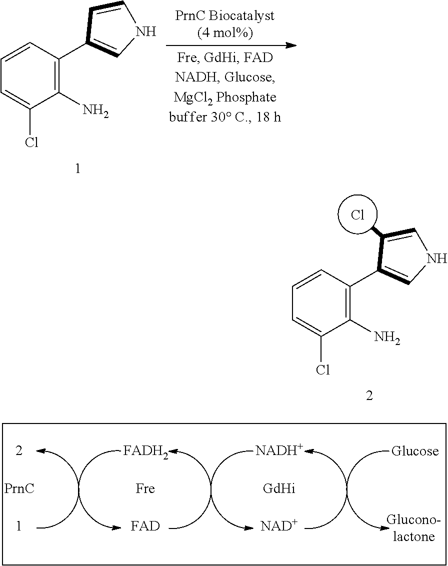

| Entry | Variation from standard conditions | conversionª of 2 (%) |
|---|---|---|
| 1. | 2 mol% FAD (10x), no GdHi & glucose | 18 |
| 2. | 2 mol% FAD (10x), GdHi regen | 16 |
| | None | 91^{b} |
| 4. | 1 mol% of Fre (2x) | 72 |
| 5. | 0.05 Equiv NADH (Less 100x) | 32 |
| 6. | 0.05 Equiv NADH (Less 100x) + 8 mol% PrnC (2x) | 53 |
| 7. | 0.05 Equiv NADH (Less 100x) in Tris HCI buffer | 49 |

^{a}Conversion is calculated based on calibration curves of product (2) and starting material (1).
^{b}Conditions: PrnC Biocatalyst (4 mol%), GdHi (0.5 mol%), FAD (0.2 mol%), Fre (0.5 mol%), NADH (5.0 equiv), Glucose (10.0 equiv), MgCl₂ (20.0 equiv), phosphate buffer (10 mM, pH 7.4)

The conditions in the table above would be referred to as the standard conditions of the present invention; i.e. flavin-dependent halogenase (4 mol %), flavin adenine dinucleotide (FAD) (0.2 mol %), E. coli flavin reductase (Fre) (0.5 mol %), nicotinamide adenine dinucleotide hydrogen (NADH) (5.0 equiv), glucose 1-dehydrogenase (GdHi) (0.5 mol %), monosaccharide (10.0 equiv), halogen (20.0 equiv), buffer (10 mM, pH 7.4).

With the purified biocatalyst PrnC enzyme in hand, a preliminary optimization study of the enzymatic assay conditions was performed on the native substrate monodechloroaminopyrrolnitrin (1). The enzyme assays were carried out for 18 h with orbital shaking at 300 rpm and a temperature of 30° C. In addition to the substrate and PrnC, the reaction mixture contained FAD (0.1 equivalents relative to PrnC), NADH and E. coli flavin reductase (Fre), needed to generate FADH₂ for the halogenation reaction. In initial assays, even using a super stoichiometric amount of NADH (5.0 equiv) (entry 1) only yielded 18% conversion to the desired product. Addition of a glucose dehydrogenase (GdHi)-NADH regeneration system also did not lead to discernable improvement (entry 2), indicating that other parameters should be evaluated.

Surprisingly, a tenfold decrease in FAD concentration (entry 3) had a major effect on the conversion yield, increasing it to 91%. The reasoning is that is that while the rate of FADH₂ formation is slower, it parallels the progressive generation of hypohalous acid that is necessary for the formation of the chloroamine intermediate involved in site-specific halogenation. Conversely, increasing the Fre concentration to maximize FADH₂ turnover (entry 4) did not produce results that were comparable to the standard conditions. A potential reason for this could be the accumulation of uncoupled FADH₂ that reacts with molecule oxygen to form H₂O₂, causing detrimental effects on the enzyme.

Despite already being present in excess (relative to FAD), conservation of relatively expensive NADH (entry 5) did not yield comparable results to standard conditions. This data indicates that a threshold amount of NADH is necessary to initiate the cascade catalytic cycle. An increase of two-fold in the biocatalyst loading (c.f. entry 5 vs entry 6) led to an almost corresponding increase in 2, possibly due to the higher coupling probability between the halogenase and FADH₂. The switch from phosphate to Tris HCl buffer (c.f. entry 5 vs entry 7) appears to provide an incremental improvement but was not pursued because of conflicts with other potential halide sources (i.e., Br—).

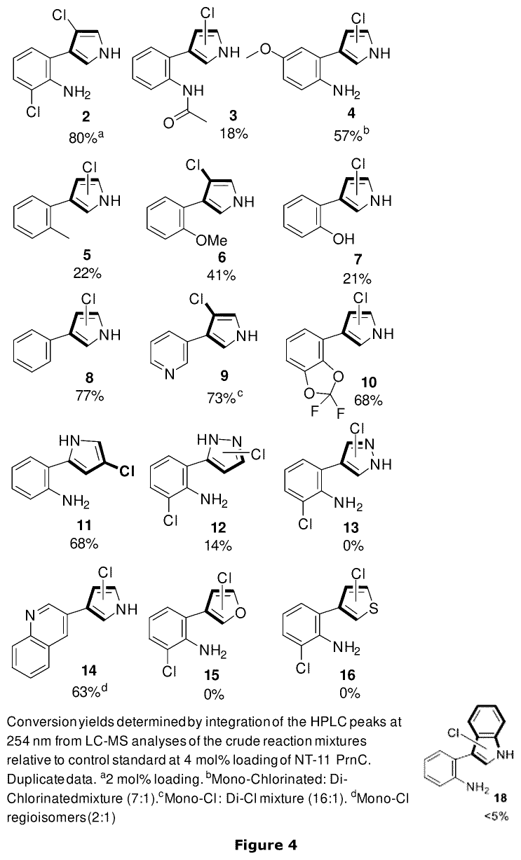

In-vitro halogenation of the native MDA substrate (2′-pyrroles) using our optimized conditions gave 55% of product 2. The catalytic efficiency (K_{cat}/Kₘ) of PrnC enzyme against MDA substrate was determined to be 4.83×10² s⁻¹ M⁻¹ (FIG. 8). A curated list of biaryl-pyrroles was assembled (FIG. 4) with respect to a) the different substituents on the adjacent phenyl group, b) the structural positional isomers of the pyrrolic fragment and c) other heterocyclic 5-membered rings. This regioselectivity of this bio-halogenation has been analogously assigned to be on the backbone of the pyrrolic fragment based on our computation model with further collaboration by 2D NMR structural confirmation on selected substrates 6, 9 and 11.

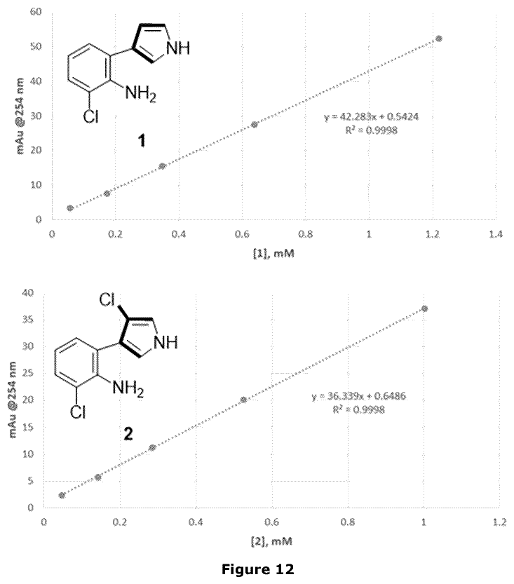

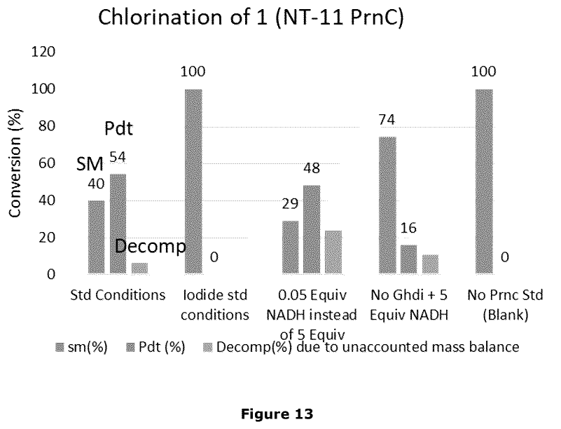

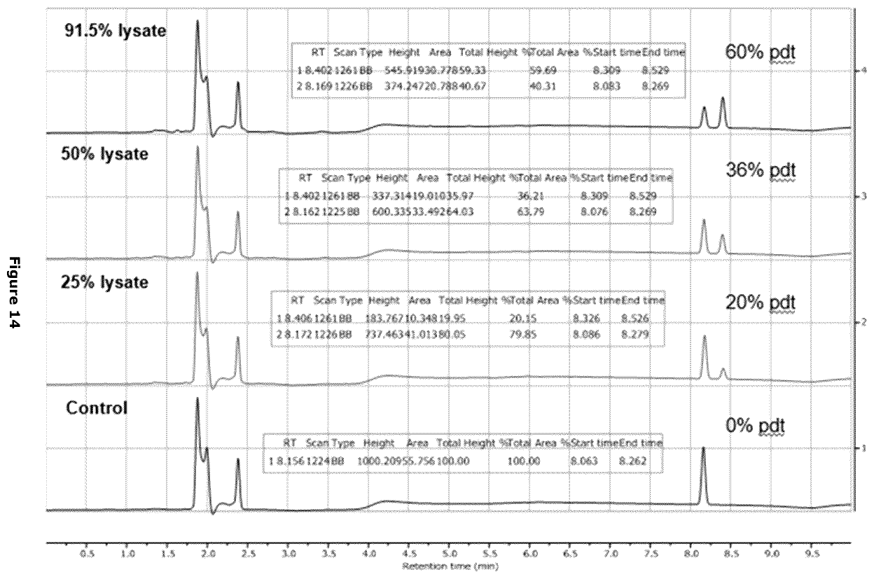

The capping of the free amino group with acetanilide in product 3 seems to significantly reduce the conversion yield (18%), which may be due to the limited space for an extra acetyl group (FIG. 12). In products 5, 6, and 7, the simultaneous substitution of the ortho-phenyl —NH₂ group and removal of the phenyl 3-Cl atom had a moderately negative impact on the conversion yields (22%, 41% and 21%). These results also confirm that prior installation of the 3-Cl atom by PrnA has no bearing on the subsequent step of halogenation.

The total removal of substituents from the aryl fragment 8 (77%) or substitution with a heterocycle pyridine 9 (73%) or quinoline 14 (63%) yielded good conversion, indicating that the hydrophobic interaction of these phenyl groups embedded in the hydrophobic patch is highly relevant (FIG. 12). These findings further suggest that the —NH₂ moiety does not appear to play an active directing function in the halogenation step.

Other bio-halogenation attempts on isosteric alternatives in the pyrrolic fragment were met with moderate success with 2′-pyrrole 11 (68%) and 5′-pyrazole 12 (14%). Other 5-membered heterocycles such as furan 15 and thiophene 16, on the other hand, were not tolerated by the PrnC enzyme. This may be hypothesized that these substrates are unable to form a hydrogen bond with the crucial residue E60, as illustrated in FIG. 2, despite having electron-rich rings that are highly susceptible to electrophilic substitution.

It is believed that in 13, the highly electron deactivated pyrazole ring coupled with unfavorable binding interaction of the Nitrogen-atom with the active residues within the enzyme binding pocket may hinder the halogenation reaction at 3′ and 5′ position.

To further demonstrate the utility of the Prnc biocatalyst on a practical target, the enzymatic halogenation sequence was applied en route to the synthesis of Fludioxonil (FIGS. 6 and 7).

### Stage 1

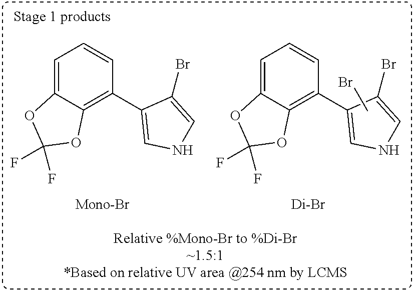

### Stage 2: (Done in 1 Pot)

Upon completion of stage 1, crude mixture was directly topped up with reagents and submitted to conditions from stage 2.

% Conversion has not been determined but a strong mass signal on −ve mode LCMS has been detected which correspond to the mass of the CN-product.

NMR spectra were recorded on Bruker Avance III 400 MHz spectrometer in CDCl3 or MeOD-d4. Data is reported in the following order: chemical shifts are given(δ); multiplicities are indicated as s (singlet), d (doublet), t (triplet), q (quartet) and m (multiplet).

Chemicals and anhydrous solvents were obtained from Sigma Aldrich and were used without further purification.

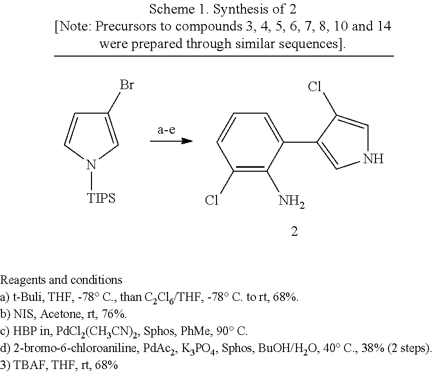

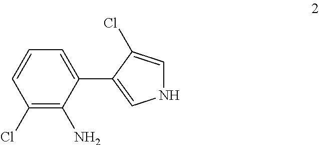 [SMILES: Nc1c(Cl)cccc1-c1c[nH]cc1Cl]

### 2-chloro-6-(4-chloro-1H-pyrrol-3-yl)aniline (2)

¹H NMR (400 MHz, CDCl₃) δ 8.30 (s, 1H), 7.24 (dd, J=8.0, 1.5 Hz, 1H), 7.09 (dd, J=7.6, 1.5 Hz, 1H), 6.86 (ddd, J=15.4, 2.9, 2.3 Hz, 2H), 6.71 (t, J=7.8 Hz, 1H), 4.16 (s, 2H). ¹³C NMR (100 MHz, CDCl₃) δ 141.7, 130.0, 128.5, 120.1, 119.4, 119.3, 117.92, 117.2, 116.2, 112.2. This is in accordance with literature data.

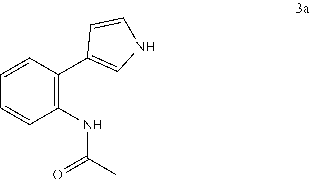

### N-(2-(1H-pyrrol-3-yl)phenyl)acetamide (3a)

¹H NMR (400 MHz, CDCl₃) δ 8.52 (s, 1H), 8.31 (d, J=8.2 Hz, 1H), 7.71 (s, 1H), 7.34-7.23 (m, 2H), 7.14-7.06 (m, 1H), 6.96-6.88 (m, 2H), 6.36 (q, J=2.4 Hz, 1H), 2.10 (s, 3H). ¹³C NMR (101 MHz, CDCl₃) δ=168.2, 135.2, 129.9, 127.3, 126.2, 124.0, 120.7, 120.7, 119.2, 116.7, 108.8, 24.8. IR (neat) 3245, 1624, 1602, 1527, 1373, 1298, 1084, 759 cm⁻¹. HRMS (ESI) calcd, for C₁₂H₁₃N₂O m/z (M+H)⁺: 201.1028, found: 201.1016.

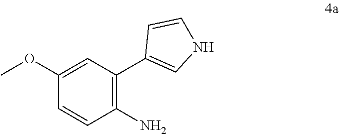

### 4-methoxy-2-(1H-pyrrol-3-yl)aniline (4a)

¹H NMR (400 MHz, CDCl₃) δ=8.36 (s, 1H), 7.02 (dt, J=2.6, 1.8 Hz, 1H), 6.94-6.84 (m, 2H), 6.75-6.64 (m, 2H), 6.46 (td, J=2.7, 1.6 Hz, 1H), 3.77 (s, 3H), 3.70 (s, 2H). ¹³C NMR (101 MHz, CDCl₃) δ=152.7, 137.5, 123.5, 122.0, 118.5, 116.7, 116.4, 115.3, 112.9, 108.7, 55.8. IR (neat) 3351, 3284, 2963, 1615, 1508, 1475, 1284, 1241, 1207, 871, 806, 746, 726 cm⁻¹. HRMS (ESI) calcd. for C₁₁H₁₃N₂O m/z (M+H)⁺: 189.1028, found: 189.1087.

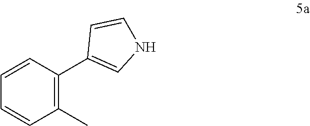

### 2-(o-tolyl)-1H-pyrrole (5a)³

¹H NMR (400 MHz, CDCl₃) δ 8.29 (s, 1H), 7.39 (dd, J=7.5, 1.7 Hz, 1H), 7.27-7.11 (m, 3H), 6.90 (dt, J=2.6, 1.8 Hz, 1H), 6.85 (td, J=2.7, 2.0 Hz, 1H), 6.42 (td, J=2.7, 1.6 Hz, 1H), 2.44 (s, 3H). ¹³C NMR (101 MHz, CDCl₃) δ=135.8, 135.3, 130.5, 129.3, 125.9, 125.8, 124.4, 117.6, 116.6, 109.5, 21.4. HRMS (ESI) calcd. for C₁₁H₁₂N m/z (M+H)⁺: 158.0961, found: 158.0970. This is in accordance with literature data.

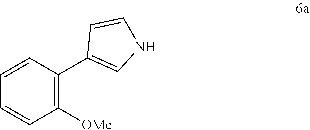 [SMILES: COc1ccccc1-c1cc[nH]c1]

### 2-(2-methoxyphenyl)-1H-pyrrole (6a)⁴

¹H NMR (400 MHz, CDCl₃) δ=8.27 (s, 1H), 7.56 (dd, J=7.6, 1.7 Hz, 1H), 7.36 (dt, J=2.7, 1.8 Hz, 1H), 7.17 (ddd, J=8.2, 7.4, 1.8 Hz, 1H), 7.03-6.91 (m, 2H), 6.84 (td, J=2.8, 2.0 Hz, 1H), 6.64 (td, J=2.8, 1.5 Hz, 1H), 3.90 (s, 3H). ¹³C NMR (101 MHz, CDCl₃) δ 156.1, 128.0, 126.3, 124.6, 120.8, 120.4, 117.9, 117.7, 111.1, 108.0, 55.4. HRMS (ESI) calcd, for C₁₁H₁₂NO m/z (M+H)⁺: 174.0913, found: 174.0919. This is in accordance with literature data.

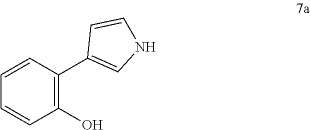 [SMILES: Oc1ccccc1-c1cc[nH]c1]

### 2-(1H-pyrrol-2-yl)phenol (7a)

¹H NMR (400 MHz, CDCl₃) δ 8.44 (s, 1H), 7.33 (dd, J=7.5, 1.8 Hz, 1H), 7.17 (ddd, J=8.1, 7.3, 1.7 Hz, 1H), 7.05 (q, J=2.0 Hz, 1H), 7.00-6.89 (m, 3H), 6.44 (td, J=2.7, 1.6 Hz, 1H), 5.60 (s, 1H). ¹³C NMR (101 MHz, CDCl₃) δ=152.7, 129.4, 127.7, 122.6, 120.5, 119.5, 119.4, 116.4, 115.2, 108.4. IR (neat) 3418, 1452, 753 cm⁻¹. HRMS (ESI) calcd, for C₁₀H₁₀NO m/z (M+H)⁺: 160.0755, found: 160.0762.

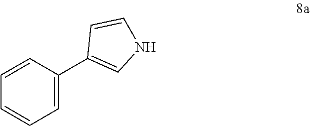 [SMILES: c1ccc(-c2cc[nH]c2)cc1]

### 3-phenyl-1H-pyrrole (8a) 5

¹H NMR (400 MHz, CDCl₃) δ=8.25 (s, 1H), 7.60-7.52 (m, 2H), 7.40-7.31 (m, 2H), 7.24-7.15 (m, 1H), 7.10 (dt, J=2.6, 1.8 Hz, 1H), 6.84 (td, J=2.7, 2.0 Hz, 1H), 6.57 (td, J=2.7, 1.6 Hz, 1H). ¹³C NMR (101 MHz, CDCl₃) δ 135.8, 128.6, 125.5, 125.3, 125.0, 118.9, 114.6, 106.6. HRMS (ESI) calcd, for C₁₀H₁₀N m/z (M+H)⁺: 144.0813, found: 144.0804. This is in accordance with literature data.

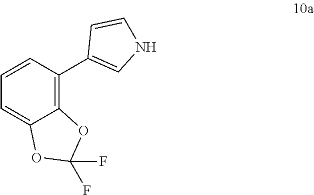 [SMILES: FC1(F)Oc2cccc(-c3cc[nH]c3)c2O1]

### 3-(2,2-difluorobenzo[d][1,3]dioxol-4-yl)-1H-pyrrole (10a)

¹H NMR (400 MHz, CDCl₃) δ 8.41 (s, 1H), 7.34 (dt, J=2.8, 1.8 Hz, 1H), 7.29 (dd, J=8.2, 1.2 Hz, 1H), 7.05 (t, J=8.0 Hz, 1H), 6.92-6.81 (m, 2H), 6.66 (td, J=2.8, 1.6 Hz, 1H). ¹³C NMR (101 MHz, CDCl₃) δ 144.0, 139.6, 131.5 (¹JC-F=254.5 Hz), 123.6, 120.6, 119.4, 118.9, 117.6, 117.4, 106.9, 105.9. IR (neat) 3410 3392 1654 1453 1231 1130 1082 880 767 723 712 cm⁻¹. HRMS (ESI) calcd, for C₁₁H₈F₂NO₂ m/z (M+H)⁺: 224.0523, found: 224.0514.

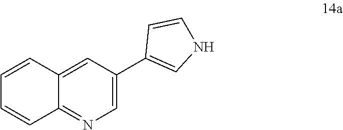 [SMILES: c1ccc2ncc(-c3cc[nH]c3)cc2c1]

### 3-(1H-pyrrol-3-yl)quinolone (14a)

¹H NMR (400 MHz, CDCl₃) δ 9.16 (d, J=2.3 Hz, 1H), 8.59 (s, 1H), 8.24-8.14 (m, 1H), 8.07 (dq, J=8.5, 0.9 Hz, 1H), 7.81 (ddt, J=8.1, 1.4, 0.5 Hz, 1H), 7.63 (ddd, J=8.4, 6.9, 1.5 Hz, 1H), 7.52 (ddd, J=8.1, 6.9, 1.2 Hz, 1H), 7.29 (dt, J=2.8, 1.8 Hz, 1H), 6.94 (td, J=2.8, 2.0 Hz, 1H), 6.70 (td, J=2.7, 1.6 Hz, 1H). ¹³C NMR (101 MHz, CDCl₃) δ 149.42, 146.55, 129.80, 129.15, 128.97, 128.55, 128.21, 127.53, 126.72, 121.71, 119.59, 115.39, 106.70. IR (neat) 3168 2924 2854 1602 1489 1339 1165 1083 902 789 748 cm⁻¹. HRMS (ESI) calcd, for C₁₃H₁₁N₂ m/z (M+H)⁺: 195.0922, found: 195.0913.

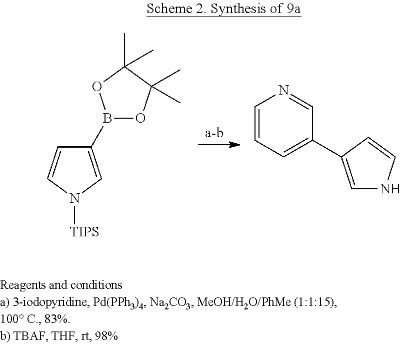

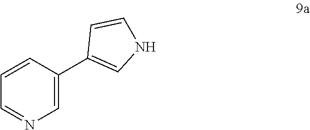 [SMILES: c1cncc(-c2cc[nH]c2)c1]

### 3-(1H-pyrrol-3-yl)pyridine (9a)⁶

¹H NMR (400 MHz, CDCl₃) δ=9.08 (s, 1H), 8.81 (dd, J=2.3, 0.9 Hz, 1H), 8.40 (dd, J=4.8, 1.6 Hz, 1H), 7.80 (ddd, J=7.9, 2.3, 1.6 Hz, 1H), 7.30-7.22 (m, 1H), 7.15 (dt, J=2.7, 1.8 Hz, 1H), 6.88 (td, J=2.7, 1.9 Hz, 1H), 6.55 (td, J=2.7, 1.6 Hz, 1H). ¹³C NMR (101 MHz, CDCl₃) δ 146.6, 146.4, 132.4, 131.9, 123.6, 121.3, 119.5, 115.2, 106.3, 77.3. HRMS (ESI) calcd, for C₉H₉N₂ m/z (M+H)⁺: 145.0766, found: 145.0754. This is in accordance with literature data.

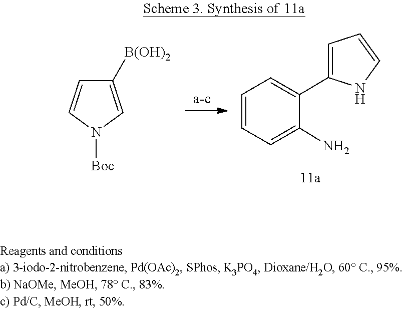 [SMILES: CC(C)(C)OC(=O)n1ccc(B(O)O)c1 ; Nc1ccccc1-c1ccc[nH]1]

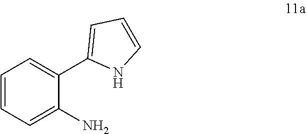 [SMILES: Nc1ccccc1-c1ccc[nH]1]

### 2-(1H-Pyrrol-2-yl)aniline (11a) 7

¹H NMR (400 MHz, CDCl₃) δ=8.60 (s, 1H), 7.26 (dd, J=7.7, 1.5 Hz, 1H), 7.10 (ddd, J=7.9, 7.4, 1.6 Hz, 1H), 6.88 (td, J=2.7, 1.5 Hz, 1H), 6.86-6.74 (m, 2H), 6.43 (ddd, J=3.4, 2.6, 1.5 Hz, 1H), 6.33 (dt, J=3.4, 2.6 Hz, 1H), 3.96 (s, 2H). ¹³C NMR (101 MHz, CDCl₃) δ 143.4, 129.6, 128.5, 127.9, 119.7, 119.2, 118.0, 116.5, 109.4, 107.4. HRMS (ESI) calcd, for C₁₀H₁₁N₂ m/z (M+H)⁺: 159.0922, found: 159.0911. This is in accordance with literature data.

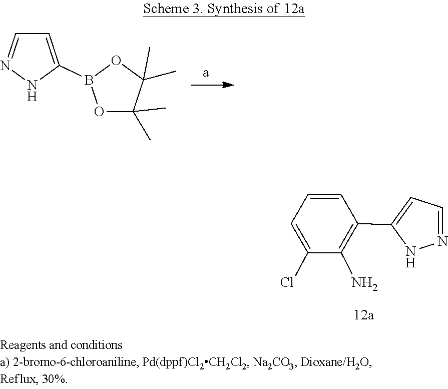

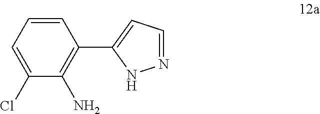 [SMILES: Nc1c(Cl)cccc1-c1ccn[nH]1]

### 2-chloro-6-(1H-pyrazole-5-yl)aniline (12a)

¹H NMR (400 MHz, CDCl₃) δ=7.62 (d, J=2.5 Hz, 1H), 7.46 (dd, J=7.8, 1.5 Hz, 1H), 7.24 (dd, J=7.9, 1.5 Hz, 1H), 6.74-6.62 (m, 2H), 5.96 (s, 2H). ¹³C NMR (101 MHz, CDCl₃) δ=151.4, 141.4, 129.3, 128.6, 126.8, 120.1, 117.3, 116.9, 103.6. IR (neat) 3426 3310 2924 1611 1458 1074 1045 747 cm⁻¹. HRMS (ESI) calcd, for C₉H₉ClN₃ m/z (M+H)⁺: 194.0485, found: 194.0485.

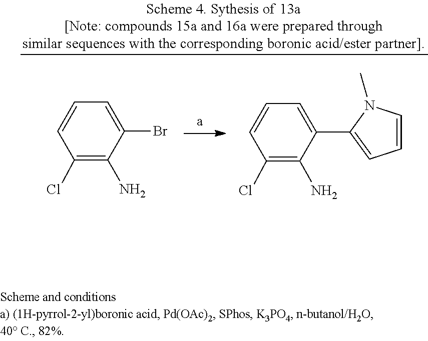

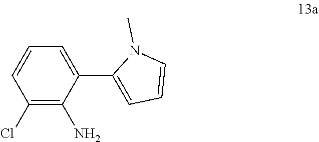

### 2-chloro-6-(1-methyl-1H-pyrrol-2-yl)aniline (13a)

¹H NMR (400 MHz, CDCl₃) δ 7.29-7.27 (m, 1H), 7.02 (dd, J=7.6, 1.5 Hz, 1H), 6.75 (dd, J=2.7, 1.7 Hz, 1H), 6.70 (dd, J=8.0, 7.5 Hz, 1H), 6.23 (dd, J=3.5, 2.7 Hz, 1H), 6.18 (dd, J=3.5, 1.8 Hz, 1H), 4.19 (s, 2H), 3.48 (s, 3H). ¹³C NMR (101 MHz, CDCl₃) δ 142.7, 130.2, 129.8, 129.2, 123.1, 119.9, 119.3, 117.8, 109.1, 107.9, 34.4. IR (neat) 3476 3374 3103 2941 2295 1858 1610 1586 1486 1451 1425 1408 1358 1309 1250 10731054 989 888 776 755 736 715 cm⁻¹. HRMS (ESI) calcd, for C₁₁H₁₁ClN₂ m/z (M+H)⁺: 207.0689, found: 207.0682.

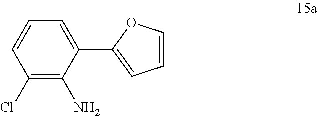 [SMILES: Nc1c(Cl)cccc1-c1ccco1]

### 2-chloro-6-(furan-2-yl)aniline (15a)

¹H NMR (400 MHz, CDCl₃) δ 7.52 (dd, J=1.9, 0.8 Hz, 1H), 7.37 (dd, J=7.8, 1.5 Hz, 1H), 7.23 (dd, J=7.9, 1.5 Hz, 1H), 6.71 (t, J=7.9 Hz, 1H), 6.62 (dd, J=3.4, 0.8 Hz, 1H), 6.52 (dd, J=3.4, 1.9 Hz, 1H), 4.85 (s, 2H). ¹³C NMR (101 MHz, CDCl₃) δ 152.8, 141.8, 140.0, 128.9, 126.3, 120.6, 118.2, 117.4, 111.6, 107.4. IR (neat) 3468 3374 1615 1499 1463 1447 1372 1317 1271 1244 1213 1152 1075 1014 905 885 802 772 725 cm⁻¹. HRMS (ESI) calcd, for C₁₀H₈ClNO m/z (M+H)⁺: 194.0373, found: 194.0367.

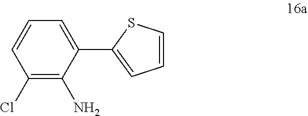 [SMILES: Nc1c(Cl)cccc1-c1cccs1]

### 2-chloro-6-(thiophen-2-yl)aniline (16a)

¹H NMR (400 MHz, CDCl₃) δ 7.37 (dd, J=5.2, 1.2 Hz, 1H), 7.26-7.20 (m, 2H), 7.17 (dd, J=7.7, 1.5 Hz, 1H), 7.13 (dd, J=5.2, 3.5 Hz, 1H), 6.71 (t, J=7.8 Hz, 1H), 4.43 (s, 2H). ¹³C NMR (101 MHz, CDCl₃) δ 141.1, 140.3, 129.6, 129.2, 127.8, 126.4, 125.9, 121.1, 119.9, 118.3. IR (neat) 3421 3325 1893 1843 1793 1614 1452 1348 1304 1268 1233 1197 1149 1069 1044 972 949 850 825 765 725 736 688 cm⁻¹. HRMS (ESI) calcd, for C₁₀H₈ClNS m/z (M+H)⁺: 210.0144, found: 210.0138.

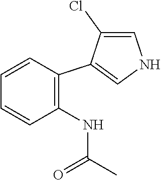 [SMILES: CC(=O)Nc1ccccc1-c1c[nH]cc1Cl]

### N-(2-(4-chloro-1H-pyrrol-3-yl)phenyl)acetamide (3)

¹H NMR (400 MHz, Methanol-d4) δ 8.55 (s, 1H), 7.70 (d, J=8.0 Hz, 1H), 7.31 (d, J=6.8 Hz, 1H), 7.29 (t, J=7.0 Hz, 1H), 7.19 (t, J=7.5 Hz, 1H), 6.85 (d, J=2.4 Hz, 1H), 6.79 (d, J=2.3 Hz, 1H), 2.04 (s, 3H). ¹³C NMR (101 MHz, Methanol-D4) δ 172.1, 136.7, 132.5, 129.2, 128.3, 126.3, 125.8, 119.6, 118.7, 117.3, 112.1, 23.5. HRMS m/z (ESI) cald for C₁₂H₁₂ClN₂₀ ([M+H]⁺) 235.0638, found: 235.0637.

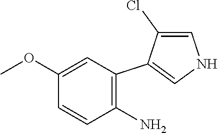

### 2-(4-chloro-1H-pyrrol-3-yl)-4-methoxyaniline (4)

¹H NMR (400 MHz, Methanol-d4) δ 7.40-7.32 (m, 1H), 7.09-7.02 (m, 2H), 6.98 (d, J=1.4 Hz, 2H), 3.88 (s, 3H). ¹³C NMR (101 MHz, Methanol-D4) δ 161.1, 131.7, 125.5, 123.3, 119.3, 119.1, 118.5, 117.5, 115.3, 112.1, 56.5. HRMS m/z (ESI) cald for C₁₁H₁₂N₂OCl ([M+H]⁺) 223.0638, found: 223.0669.

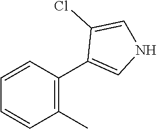

### 3-chloro-4-(o-tolyl)-1H-pyrrole (5)

¹H NMR (400 MHz, Methylene Chloride-d2) δ 8.36 (s, 1H), 7.31-7.14 (m, 4H), 6.85 (q, J=2.8 Hz, 1H), 6.72 (q, J=2.9 Hz, 1H), 2.28 (d, J=2.8 Hz, 3H). ¹³C NMR (101 MHz, CD₂Cl₂) δ 137.5, 133.2, 131.1, 130.0, 127.3, 125.3, 122.4, 117.0, 115.4, 112.1, 20.1. HRMS m/z (ESI) cald for C₁₁H₁₁ClN ([M+H]⁺) 192.0580, found: 192.0574.

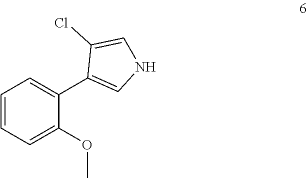

### 3-chloro-4-(2-methoxyphenyl)-1H-pyrrole (6)

Biotransformation was prepared as per the general preparative PrnC-Cell lysate chlorination method detailed above to overall volume of 20 mL. ¹H NMR (400 MHz, Methanol-d4) δ 7.58 (ddd, J=7.6, 1.8, 0.4 Hz, 1H), 7.44-7.35 (m, 1H), 7.21-7.14 (m, 1H), 7.11 (td, J=7.5, 1.2 Hz, 1H), 7.04 (d, J=2.4 Hz, 1H), 6.93 (d, J=2.4 Hz, 1H), 3.97 (s, 3H). HRMS m/z (ESI) cald for C₁₁H₁₁ClNO ([M+H]⁺) 208.0529, found: 208.0523.

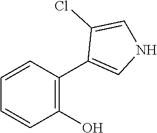 [SMILES: Oc1ccccc1-c1c[nH]cc1Cl]

### 2-(4-chloro-1H-pyrrol-3-yl)phenol (7)

¹H NMR (400 MHz, Methanol-d4) δ 7.41 (dd, J=7.6, 1.7 Hz, 1H), 7.11 (ddd, J=8.1, 7.3, 1.7 Hz, 1H), 6.97 (d, J=2.1 Hz, 1H), 6.88 (dd, J=8.2, 1.2 Hz, 1H), 6.86 (td, J=7.4, 1.3 Hz, 1H), 6.81 (d, J=2.4 Hz, 1H). ¹³C NMR (101 MHz, Methanol-D4) δ 155.9, 132.4, 128.7, 122.8, 120.6, 119.6, 118.6, 116.9, 116.7, 112.2, 49.3. HRMS m/z (ESI) cald for C₁₀H₉ClNO ([M+H]⁺) 194.0373, found: 194.0365.

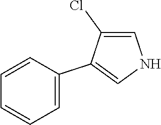 [SMILES: Clc1c[nH]cc1-c1ccccc1]

### 3-chloro-4-phenyl-1H-pyrrole (8)

¹H NMR (400 MHz, Methylene Chloride-d2) δ 8.42 (s, 1H), 7.61-7.54 (m, 2H), 7.41-7.33 (m, 2H), 7.29-7.22 (m, 1H), 6.95-6.89 (m, 1H), 6.85 (t, J=2.6 Hz, 1H). ¹³C NMR (101 MHz, CD₂Cl₂) δ 133.9, 128.4, 128.4, 127.6, 127.6, 126.4, 122.4, 116.7, 116.3, 110.6. HRMS m/z (ESI) cald for C₁₀H₉ClN ([M+H]⁺) 178.0424, found: 178.0417.

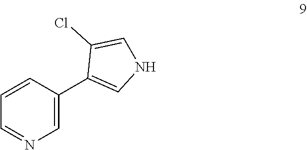 [SMILES: Clc1c[nH]cc1-c1cccnc1]

### 3-(4-chloro-1H-pyrrol-3-yl)pyridine (9)

Biotransformation was prepared as per the general preparative PrnC-Cell lysate chlorination method detailed above to overall volume of 20 mL. ¹H NMR (400 MHz, Methanol-d4) δ 8.92 (dd, J=2.3, 0.9 Hz, 1H), 8.55 (dd, J=4.9, 1.6 Hz, 1H), 8.23 (ddd, J=8.0, 2.3, 1.6 Hz, 1H), 7.60 (ddd, J=8.0, 4.9, 0.9 Hz, 1H), 7.23 (d, J=2.3 Hz, 1H), 7.04 (d, J=2.4 Hz, 1H). ¹³C NMR (101 MHz, Methanol-d₄) δ 149.2, 147.8, 137.4, 133.6, 125.9, 119.7, 119.3, 118.9, 111.6, 49.9. HRMS m/z (ESI) cald for C₉H₈N₂Cl [M+H]⁺179.0371, found: 179.0390.

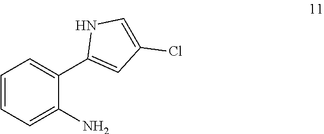 [SMILES: Nc1ccccc1-c1cc(Cl)c[nH]1]

### 2-(4-chloro-1H-pyrrol-2-yl)aniline (11)

Biotransformation was prepared as per the general preparative PrnC-Cell lysate chlorination method detailed above to overall volume of 20 mL. ¹H NMR (400 MHz, Methanol-d₄) δ 7.34-7.25 (m, 2H), 6.99 (dd, J=8.5, 1.2 Hz, 1H), 6.95 (d, J=3.0 Hz, 1H), 6.91 (td, J=7.6, 1.2 Hz, 1H), 6.32 (d, J=3.0 Hz, 1H), 5.67 (s, 1H), 4.74 (s, 2H). ¹³C NMR (101 MHz, Methanol-D4) δ 147.7, 133.0, 130.9, 127.3, 119.8, 119.7, 119.5, 117.9, 111.5, 110.5. HRMS m/z (ESI) cald for C₁₀H₁₀ClN₂ ([M+H]⁺) 193.527, found: 193.0535.

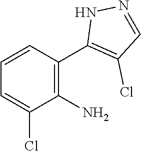 [SMILES: Nc1c(Cl)cccc1-c1[nH]ncc1Cl]

### 2-chloro-6-(4-chloro-1H-pyrazol-5-yl)aniline (12)

¹H NMR (400 MHz, Methylene Chloride-d2) δ 7.76 (s, 1H), 7.59 (dd, J=7.8, 1.5 Hz, 1H), 7.33 (dd, J=7.9, 1.4 Hz, 1H), 6.77 (t, J=7.8 Hz, 1H). ¹³C NMR (101 MHz, CD₂Cl₂) δ 144.5, 142.0, 130.2, 129.6, 128.6, 119.8, 117.1, 115.7, 109.1. HRMS m/z (ESI) cald for C₉H₈Cl₂N₃ ([M+H]⁺) 228.0095, found: 228.0098.

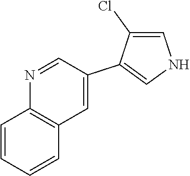 [SMILES: Clc1c[nH]cc1-c1cnc2ccccc2c1]

### 3-(4-chloro-1H-pyrrol-3-yl) quinoline

¹H NMR (400 MHz, Methylene Chloride-d2) δ 9.10 (d, J=2.3 Hz, 1H), 8.77 (s, 2H), 8.43 (d, J=2.3 Hz, 1H), 8.07 (d, J=8.5 Hz, 1H), 7.87 (dd, J=8.2, 1.4 Hz, 1H), 7.68 (ddd, J=8.5, 6.9, 1.5 Hz, 1H), 7.56 (ddd, J=8.1, 6.8, 1.3 Hz, 1H), 7.13 (t, J=2.8 Hz, 1H), 6.95 (t, J=2.6 Hz, 1H). ¹³C NMR (101 MHz, CD₂Cl₂) δ 151.0, 147.2, 133.2, 129.6, 129.3, 128.6, 128.4, 127.7, 127.3, 119.6, 117.8, 117.4, 111.5. HRMS m/z (ESI) cald for C₁₃H₁₀ClN₂ ([M+H]⁺) 229.0533, found: 227.0572.

### Comparison of Enzymatic Chlorination Vs Synthetic Methods (Reactivity and Selectivity):

Synthetic Chlorination Procedures: To demonstrate and contrast the enzymatic efficiency with commonly routinely employed protocols, chlorination was attempted using these synthetic reagents.

Three substrates 1, 11a and 14a were selected based on structural diversity in their ring sizes, electronics and substitution position with respect to the pyrrolic fragment. The chlorination procedures tested were a) N-chlorosuccinimide (NCS) in MeCN and b) sodium hypochlorite (NaOCl) prepared from Sodium Hypochlorite Pentahydrate (NaOCl·5H₂O) in aqueous buffer (50 mM sodium phosphate buffer, pH 7.4). Initial attempts to promote mono-chlorination reactions were attempted using benign conditions with only 1.2 equivalent of the chlorinating source at room temperature, stirring at 600 rpm for 18 hours. Reactions were quenched with an equal volume of MeOH and analyzed by LCMS.

Additional investigation was conducted under more accelerating conditions in light of the initial low conversion observed with only 10 equivalents of the chlorinating source at 50° C., stirring at 600 rpm for 2 days. Compared to the presently disclosed method, synthetic reactions require heating at elevated temperature (vs room temperature), the using of organic solvent (vs water), and the formation of a mixture of isomers (vs 1 major isomer).

### Synthesis of Substrate 10a and Chlorinated Fludioxonil Analog 10

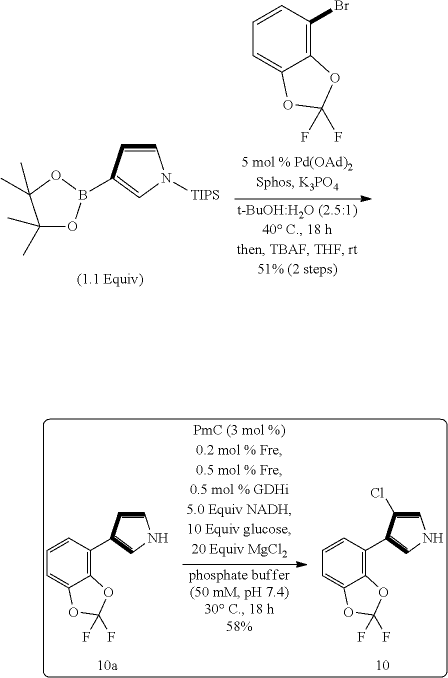

4-bromo-2,2-difluorobenzo[d][1,3]dioxole (237 mg, 0.14 mL, 1 mmol), N-TIPS pyrrole-3-boronic acid pinacol ester (384 mg, 1.1 mmol, 1.1 eq), Palladium acetate (12 mg, 5 mol %), SPhos (33 mg, 8 mol %) and K₃PO₄ (0.38 g, 1.8 mmol) were added to a schlenk tube containing degassed n-BuOH:H₂O (2.5:1, 3 ml). The mixture was stirred at 40° C. for 18 hours. Upon complete conversion of starting material by TLC, water (5 mL) was added to the mixture and extracted with (5×3 mL) of ethyl acetate. The organic layers were combined, filtered through a plug of celite and dried over anhydrous sodium sulphate. Concentration over reduced pressure afforded a crude colorless oil (341 mg, 90%) which was used directly in the next step without purification. ¹H NMR (400 MHz, CDCl₃) δ 7.30-7.26 (m, 2H), 7.03 (t, J=8.0, 1H), 6.88-6.77 (m, 2H), 6.77-6.66 (m, 1H), 1.54-1.45 (m, 3H), 1.14 (d, J=7.5, 18H).

3-(2,2-difluorobenzo[d][1,3]dioxol-4-yl)-1H-pyrrole (341 mg, 0.9 mmol) was dissolved in THF (5.0 ml) under nitrogen. TBAF (1.0 M in THF, 1.6 ml, 1.8 eq) was added and the mixture was stirred at room temperature for 1 hour. Upon complete conversion of starting material by TLC, the mixture was evaporated to dryness. The resultant residue was directly subjected to purification by silica chromatography. Side product silanol and unreacted starting material were removed by 10:1 of (hexane:ethyl acetate) and the product 10a (113 mg, 57%) was eluted using 3:1 of (hexane:ethyl acetate) as a red oil. ¹H NMR (400 MHz, CDCl₃) δ 8.41 (s, 1H), 7.34 (dt, J=2.8, 1.8 Hz, 1H), 7.29 (dd, J=8.2, 1.2 Hz, 1H), 7.05 (t, J=8.0 Hz, 1H), 6.92-6.81 (m, 2H), 6.66 (td, J=2.8, 1.6 Hz, 1H). ¹³C NMR (101 MHz, CDCl₃) δ 144.0, 139.6, 131.5 (¹JC-F=254.5 Hz), 123.6, 120.6, 119.4, 118.9, 117.6, 117.4, 106.9, 105.9. IR (neat) 3410 3392 1654 1453 1231 1130 1082 880 767 723 712 cm⁻¹. HRMS (ESI) calcd, for C₁₁H₈F₂NO₂ m/z (M+H)⁺: 224.0523, found: 224.0514.

3-chloro-4-(2,2-difluorobenzo[d][1,3]dioxol-4-yl)-1H-pyrrole (10): In a solution containing 10a (0.5 mM), MgCl₂ (10 mM), glucose (5.0 mM), FAD (1.0 μM), NT-11 PrnC (12.5 μM), Fre (2.5 μM) and Gdhi (2.5 μM) in 50 mM potassium phosphate buffer, NADH (2.5 mM) was added to a total volume of 8.0 mL. After an overnight incubation of 30° C. and orbital shaking at 350 rpm, reactions were quenched with an equivalent volume of MeOH, pelleted by centrifugation (15000 rpm for 10 min) and the supernatant extracted with (3×10 mL) of ethyl acetate. The precipitated solids were vortex, sonicated and extracted with additional (3×10 mL) of MeOH. The combined organic layers were filtered through a plug of celite and dried over anhydrous sodium sulphate. Concentration over reduced pressure afforded a crude brown oil which was subjected over preparatory thin-layer chromatography using 3:1 of (hexane:ethyl acetate) to afford (0.6 mg, 58%) of 10 as a reddish-brown oil. ¹H NMR (400 MHz, Methanol-d₄) δ 7.57 (dd, J=8.1, 1.2 Hz, 1H), 7.14 (t, J=8.1 Hz, 1H), 7.09 (d, J=2.3 Hz, 1H), 7.02 (dd, J=8.0, 1.1 Hz, 1H), 6.86 (d, J=2.3 Hz, 1H). ¹³C NMR (101 MHz, Methanol-D4) δ 144.9, 141.4, 132.7 (¹JC-F=251.6 Hz), 124.7, 124.3, 119.9, 119.6, 118.2, 114.4, 111.0, 107.8. HRMS m/z (ESI) cald for C₁₁H₅ClF₂NO₂ ([M+H]⁺) 255.9982, found: 255.9989.

In conclusion, we have successfully enhanced the heterologously expressed of PrnC as soluble proteins by attaching an 11-amino acid long NT-11 tag. This modification enables the smooth scale up and easy isolation of the purified PrnC enzyme which has been a re-occurring issue with traditional standard protocols. Yields up to 9 mg/L can be routinely obtained as compared to the <0.5 mg/L without the tag (>18-fold improvement) To the best of our knowledge, this is the first time the FAD-dependent halogenase PrnC has been isolated and applied to a mini library of free-standing pyrrolic analogs. This is a mild, green approach to access a unique C-3 halogenation of the pyrrolic substrate scaffold under aqueous buffer conditions. The bio halogenation reaction was found to be highly selective for the backbone position of the pyrrole fragment, with a broad tolerance for its appended aryl group. The key residues that are responsible for PrnC's catalytic activity have been identified and proposed by molecular docking studies and mutagenesis experiments. This ability to introduce a halide handle enables further late-stage functionalization of the resulting product to more complex molecules.

It will be appreciated that many further modifications and permutations of various aspects of the described embodiments are possible. Accordingly, the described aspects are intended to embrace all such alterations, modifications, and variations that fall within the spirit and scope of the appended claims.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A method of halogenating a 2′, 3′ and/or 5′ substituted N-heteroaryl or a derivative thereof, comprising:
a) contacting the N-heteroaryl or derivative thereof with a halogen and a flavin-dependent halogenase under co-enzyme regeneration conditions in order for the halogen to be substituted at a 4′ position on the N-heteroaryl or derivative thereof;
wherein the co-enzyme regeneration conditions comprises flavin adenine dinucleotide (FAD) at about 0.01 equivalents to about 0.2 equivalents relative to a flavin-dependent halogenase concentration;
E. coli flavin reductase (Fre) at about 0.1 equivalents to about 0.5 equivalents relative to a flavin-dependent halogenase concentration;
nicotinamide adenine dinucleotide hydrogen (NADH) at about 2 equivalents to about 250 equivalents relative to a flavin-dependent halogenase concentration;
glucose 1-dehydrogenase (GdHi) at about 0.1 equivalents to about 0.5 equivalents relative to a flavin-dependent halogenase concentration;
monosaccharide at about 5 equivalents to about 500 equivalents relative to a flavin-dependent halogenase concentration.

2. The method according to claim 1, wherein the co-enzyme regeneration conditions comprises NADH at about 0.05 equivalents to about 5 equivalents relative to a 2′, 3′ and/or 5′ substituted N-heteroaryl or a derivative thereof concentration; and/or
wherein the co-enzyme regeneration conditions comprises monosaccharide at about 5 equivalents to about 20 equivalents relative to a 2′, 3′ and/or 5′ substituted N-heteroaryl or a derivative thereof concentration.

3. (canceled)

4. The method according to claim 1, wherein a ratio of FAD:Fre:GDH is about 1:2.5:2.5; and/or
wherein a ratio of FAD:Fre:NADH:GDH is about 1:2.5:2500:2.5.

5. (canceled)

6. The method according to claim 1, wherein the co-enzyme regeneration conditions further comprises a halogen at about 10 equivalents to about 30 equivalents relative to a 2′, 3′ and/or 5′ substituted N-heteroaryl or a derivative thereof concentration.

7. The method according to claim 1, wherein the halogen is derived from a halide, the halide selected from Cl⁻ or Br⁻.

8. The method according to claim 1, wherein the co-enzyme regeneration conditions further comprises a phosphate buffer or a Tris HCl buffer.

9. The method according to claim 1, wherein the flavin-dependent halogenases is monodechloroaminopyrrolnitrin halogenase (PrnC).

10. The method according to claim 1, wherein the flavin-dependent halogenases comprises a N-terminal 11 amino acid solubility tag;
wherein the N-terminal 11 amino acid solubility tag is derived from a first 11 amino acid residues within a N-terminal N-half domain of a duplicated carbonic anhydrase (dCA) from Dunaliella species.

11. (canceled)

12. The method according to claim 10, wherein the 11 amino acid solubility tag has an amino acid sequence of VSEPHDYNYEK.

13. The method according to claim 1, wherein the N-heteroaryl or derivative thereof is a 5 membered N-heteroaryl or derivative thereof.

14. The method according to claim 1, wherein the substituent on the 2′, 3′ and/or 5′ substituted N-heteroaryl or a derivative thereof is each selected from optionally substituted aryl or optionally substituted heteroaryl.

15. The method according to claim 1, wherein the N-heteroaryl or derivative thereof is a compound of formula (I) 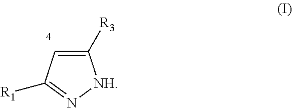
wherein X is selected from CR₂ or N;
R₁ is H, optionally substituted aryl, or optionally substituted heteroaryl;
R₂ is H, optionally substituted aryl, or optionally substituted heteroaryl;
R₃ is H, optionally substituted aryl, or optionally substituted heteroaryl;
wherein at least one of R₁, R₂ and R₃ is optionally substituted aryl or optionally substituted heteroaryl; or
R₁ and R₂ are linked to form optionally substituted aryl, or optionally substituted heteroaryl.

16. (canceled)

17. The method according to claim 15, wherein when X is CR₂, R₂ is H and R₃ is H.

18. The method according to claim 15, wherein when X is N, R₁ is H.

19. The method according to claim 1, wherein the N-heteroaryl or derivative thereof is selected from:

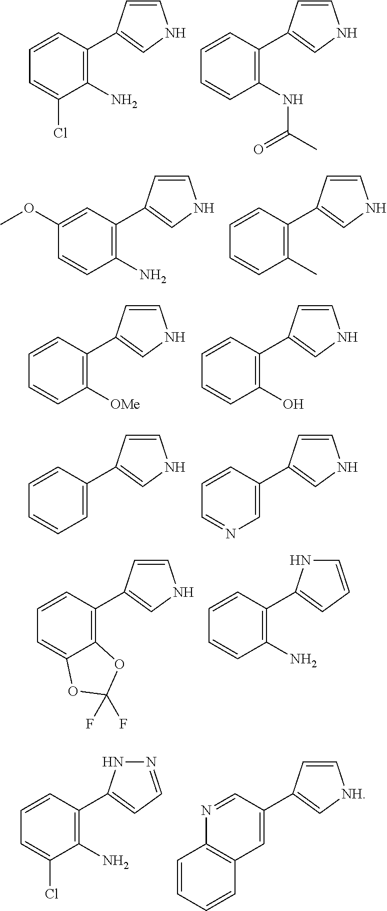

20. The method according to claim 1, wherein the halogenated N-heteroaryl or derivative thereof is (where X₁ represents halo):

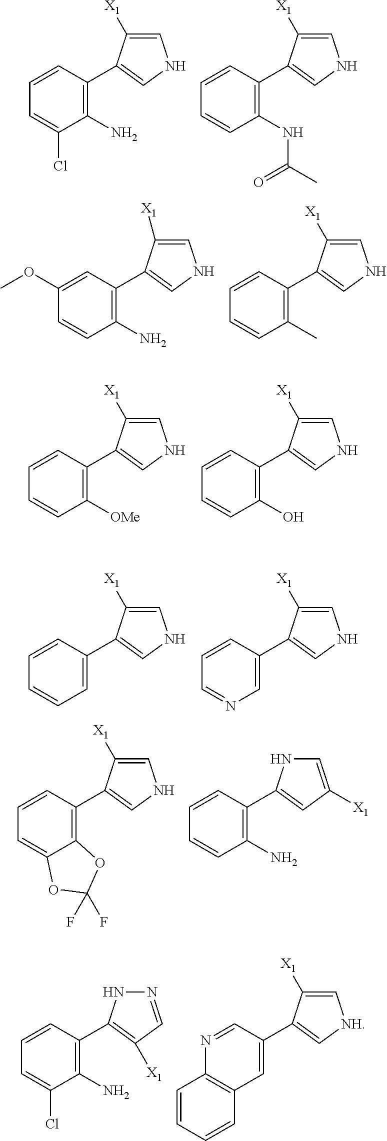

21. The method according to claim 1, wherein the method is characterised by at least one of the following:
a) a regioisomeric ratio of 4′ substitution to 2′, 3′ or 5′ substitution is about 3:1 to about 1.1:1;
b) a ratio of 4′ monohalogenation to 2′, 4′ dihalogenation, 3′, 4′ dihalogenation, and/or 4′, 5′ dihalogenation of about 20:1 to about 4:1;
c) a K_{cat} of about 7×10⁻³ s⁻¹ to about 8×10⁻³ s⁻¹;
d) a Kₘ of about 1×10⁻⁵ M to about 2×10⁻⁵ M; and
e) a K_{cat}/Kₘ of about 4×10² s⁻¹ M⁻¹ to about 5×10² s⁻¹ M⁻¹.

22. (canceled)

23. (canceled)

24. (canceled)

25. (canceled)

26. A method of producing a flavin-dependent halogenase, comprising:
a) expressing the flavin-dependent halogenase in a cell;
wherein the flavin-dependent halogenase comprises an 11 amino acid solubility tag at a N-terminus thereof and a His6 tag at a C-terminus thereof.

27. The method according to claim 26, further comprising a step of purifying the flavin-dependent halogenase by metal affinity chromatography.

28. A method of synthesising Fludioxonil, comprising:
a) contacting a compound of formula (II) or derivative thereof with a halogen and a flavin-dependent halogenase under co-enzyme regeneration conditions in order for the halogen to be substituted at a 4′ position on the compound or derivative thereof; 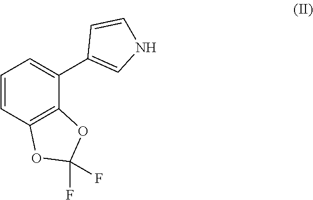 [SMILES: FC1(F)Oc2cccc(-c3cc[nH]c3)c2O1]
wherein the co-enzyme regeneration conditions comprises flavin adenine dinucleotide (FAD) at about 0.01 equivalents to about 0.2 equivalents relative to a flavin-dependent halogenase concentration;
E. coli flavin reductase (Fre) at about 0.1 equivalents to about 0.5 equivalents relative to a flavin-dependent halogenase concentration;
nicotinamide adenine dinucleotide hydrogen (NADH) at about 2 equivalents to about 250 equivalents relative to a flavin-dependent halogenase concentration;
glucose 1-dehydrogenase (GdHi) at about 0.1 equivalents to about 0.5 equivalents relative to a flavin-dependent halogenase concentration;
monosaccharide at about 5 equivalents to about 500 equivalents relative to a flavin-dependent halogenase concentration; and
b) contacting the 4′ halogenated compound of step a) with a palladium precatalyst and a cyanide precursor.


SEQUENCE LISTING (10 sequences)

SEQ ID NO: 1 - DNA, 35 bp, synthetic construct
  source 1..35: mol_type = other DNA
cgaaagttccatcccggcgacttcgttgatgaatc

SEQ ID NO: 2 - DNA, 35 bp, synthetic construct
  source 1..35: mol_type = other DNA
gattcatcaacgaagtcgccgggatggaactttcg

SEQ ID NO: 3 - DNA, 36 bp, synthetic construct
  source 1..36: mol_type = other DNA
catcgtcaaccggaattgcgcgtaatttcggctttg

SEQ ID NO: 4 - DNA, 36 bp, synthetic construct
  source 1..36: mol_type = other DNA
caaagccgaaattacgcgcaattccggttgacgatg

SEQ ID NO: 5 - DNA, 35 bp, synthetic construct
  source 1..35: mol_type = other DNA
gcttccctggggacctgcgtcacattattatcgtc

SEQ ID NO: 6 - DNA, 35 bp, synthetic construct
  source 1..35: mol_type = other DNA
gacgataataatgtgacgcaggtccccagggaagc

SEQ ID NO: 7 - DNA, 42 bp, synthetic construct
  source 1..42: mol_type = other DNA
ggagatataccatggtaagtgaaccccacgactataattatg

SEQ ID NO: 8 - DNA, 40 bp, synthetic construct
  source 1..40: mol_type = other DNA
gtggtggtgctcgagttttttcagcgctaatccaatacgc

SEQ ID NO: 9 - DNA, 1776 bp, synthetic construct
  source 1..1776: mol_type = other DNA
atggtaagtgaaccccacgactataattatgagaaagctagcgcgtctatgacacaaaaatctcccgccaacgagcacgattcaaatcactttgatgtcattattctgggatcgggcatgtcaggtacgcagatgggcgctattttggctaaacaacaatttcgtgtgttaattattgaagagtccagccacccacgttttaccattggcgaaagttccatcccggaaacttcgttgatgaatcgtatcatcgcagaccgctatggtatcccagaactggaccatattacctccttttatagcacacaacgttacgtcgcatcgtcaaccggaattaaacgtaatttcggctttgtatttcacaaaccaggtcaagagcacgatcctaaggaattcacacagtgtgtgattccggagcttccctggggacctgaatcacattattatcgtcaggacgtagatgcatatttattgcaggcggcgatcaagtacgggtgtaaagtgcaccaaaagacaactgtgacggagtatcatgctgataaggatggcgtagcggtcacaacagctcaaggggaacgcttcacaggtcgctacatgatcgattgcggtggaccccgcgcgcccttggctactaaattcaagttacgtgaagagccatgccgttttaaaacgcacagccgttctctgtatactcacatgttaggggtcaagccattcgacgacatttttaaagtaaaggggcagcgctggcgttggcatgaaggaacccttcatcacatgttcgaaggaggatggctgtgggtgattccctttaataaccatccccgtagcacaaataacttagtctcggttggtttgcaacttgacccacgtgtctaccccaagaccgacatctccgctcaacaagaattcgacgagttccttgcccgttttccgtcaatcggagcccagtttcgcgacgccgttccagttcgcgattgggtgaaaactgatcgccttcaattttctagtaatgcatgcgtcggagaccgttactgtttaatgttacacgcgaacggatttattgatccgttgttttcgcgtgggcttgagaatactgcggtcacgatccacgccttagctgcgcgtcttatcaaggccctgcgcgacgatgacttctctccagaacgcttcgagtatatcgagcgtttgcaacagaagttgttggaccacaatgacgacttcgtgtcatgttgctacacagctttttcggattttcgtctttgggatgcctttcaccgcctttgggccgtggggactatccttggacaatttcgtcttgtgcaggcccacgcacgctttcgcgcatcgcgtaacgagggtgatctggatcatttagataacgacccaccctatttggggtatctgtgcgctgatatggaagagtactatcagcttttcaatgacgccaaggctgaggtggaagcagtatcagcgggacgtaagcctgccgacgaggctgccgcgcgcattcatgccttgatcgacgaacgtgacttcgccaaacctatgttcgggttcggttactgcattacaggagataaaccacagttgaataattccaagtactctttattgcctgcgatgcgcttgatgtactggacgcagacccgtgccccagcggaagtcaagaagtatttcgattataaccctatgtttgcgttgcttaaggcatatattactacgcgtattggattagcgctgaaaaaactcgagcaccaccaccaccaccactga

SEQ ID NO: 10 - protein, 591 aa, synthetic construct
MVSEPHDYNYEKASASMTQKSPANEHDSNHFDVIILGSGMSGTQMGAILAKQQFRVLIIEESSHPRFTIGESSIPETSLMNRIIADRYGIPELDHITSFYSTQRYVASSTGIKRNFGFVFHKPGQEHDPKEFTQCVIPELPWGPESHYYRQDVDAYLLQAAIKYGCKVHQKTTVTEYHADKDGVAVTTAQGERFTGRYMIDCGGPRAPLATKFKLREEPCRFKTHSRSLYTHMLGVKPFDDIFKVKGQRWRWHEGTLHHMFEGGWLWVIPFNNHPRSTNNLVSVGLQLDPRVYPKTDISAQQEFDEFLARFPSIGAQFRDAVPVRDWVKTDRLQFSSNACVGDRYCLMLHANGFIDPLFSRGLENTAVTIHALAARLIKALRDDDFSPERFEYIERLQQKLLDHNDDFVSCCYTAFSDFRLWDAFHRLWAVGTILGQFRLVQAHARFRASRNEGDLDHLDNDPPYLGYLCADMEEYYQLFNDAKAEVEAVSAGRKPADEAAARIHALIDERDFAKPMFGFGYCITGDKPQLNNSKYSLLPAMRLMYWTQTRAPAEVKKYFDYNPMFALLKAYITTRIGLALKKLEHHHHHH